(19) United States
(12) Patent Application Publication
(10) Pub. No.: US 2023/0131629 A1
(43) Pub. Date: Apr. 27, 2023

(54) System and method for non-invasive assessment of elevated left ventricular end-diastolic pressure (LVEDP)

(71) Applicant: AventuSoft, LLC, Boca Raton, FL (US)

(72) Inventors: Kan Li, Gainesville, FL (US); Kaustubh Kale, Delray Beach, FL (US)

(73) Assignee: AventuSoft, LLC, Boca Raton, FL (US)

(21) Appl. No.: 17/982,510

(22) Filed: Nov. 7, 2022

Related U.S. Application Data
(63) Continuation-in-part of application No. 16/741,740, filed on Jan. 13, 2020, now Pat. No. 11,490,849.
Application No. 16/741,740 is a continuation-in-part of application No. 15/397,138, filed on Jan. 3, 2017, now Pat. No. 10,531,839.

(51) Int. Cl. A61B 5/0205; A61B 5/02; A61B 5/00

(52) U.S. Cl. CPC A61B 5/0205; A61B 5/02028; A61B 5/7278; A61B 5/0245

(57) ABSTRACT

A system for noninvasive extraction, identification, and marking of the heart valve signals to evaluate and monitor elevated left ventricular end-diastolic pressure (LVEDP) or pulmonary capillary wedge pressure (PCWP) using at rest assessment of hemodynamic performance, based on quantitative measurements of heart and lung related parameters and cardiac events for diagnostic and therapeutic purposes includes one or more signals from one or more noninvasive sensors or transducers that measure one or more physiological effects that are correlated with cardiopulmonary functions, transmission of the data to a computing device and analysis software where a trained algorithm processes the data to determine the state or condition of elevated LVEDP or PCWP and provides an output indicative of the state or condition of the analysis. The described noninvasive cardiopulmonary health assessment and monitoring systems and methods can provide effective at-home self-assessment or an integrated telehealth remote patient monitoring (RPM) system.

### CROSS REFERENCE TO RELATED APPLICATIONS

This Application is a Continuation-in-Part of and claims priority through U.S. patent application Ser. No. 16/741,740 filed on Jan. 13, 2020, which claims priority through U.S. patent application Ser. No. 15/397,138 filed on Jan. 3, 2017 which further claims the priority benefit of Provisional Application Nos. 62/274,766, 62/274,761, 62/274,763, 62/274,765, and 62/274,770 each of which were filed on Jan. 4, 2016, the entire disclosure of each are incorporated herein by reference.

### FIELD

The embodiments herein relate generally to cardiopulmonary health monitoring and more particularly to analysis software combined with non-invasive transducers to capture cardiac timing signals along with an electrocardiogram signal for the measurement of heart functions and their related diseases.

### BACKGROUND

Heart disease is the leading cause of death accounting for more than one-third (33.6%) of all U.S. deaths. Overall cardiac health can be significantly improved by proper triage. Low invasive and non-invasive ultrasound techniques (e.g. echocardiogram) are standard procedures, but the requirement of expensive devices and skilled operators limit their applicability. The following are the various types of heart disease that can be diagnosed and treated using the separated signal, namely, Coronary artery disease, Heart murmurs and valve abnormalities, Heart failure, Heart rhythm abnormalities (arrhythmias), vascular disease, congenital heart disease, Cardiac resynchronization and Risk factor modification. A physician can work with patients to perform a comprehensive evaluation and design a personalized plan of care aimed at keeping them healthy.

The cardiopulmonary system which consists of the respiratory components, snoring components, and cardiac components, generates time events correlated to the cardiac hemodynamics during each cardiac cycle. The time events correspond to the acceleration and deceleration of blood due to abrupt mechanical opening and closing of the heart valves during the cardiac cycle and can be observed noninvasively through mechanical, electrical, and or optical sensors.

### SUMMARY

In some embodiments, a method and system for noninvasive evaluating and monitoring of a contractility feature includes obtaining one or more signals using one or more noninvasive sensors or transducers that provide a measure of one or more physiological effects that are correlated with cardiopulmonary functions, the measure based on quantitative measurements of heart and lung related parameters and cardiac events for diagnostic and therapeutic purposes, and computing, by at least one processor, an elevated left ventricular end-diastolic pressure (LVEDP) or an pulmonary capillary wedge pressure (PCWP) based on explicit or implicit time features and waveform features of the one or more signals, where the LVEDP or the PCWP are computed at a rest assessment of hemodynamic performance.

In some embodiments, the contractility feature includes cardiac time intervals, and the method further comprising calculating, by the at least one processor, cardiac time intervals based on cardiac waveform data. In some embodiments, at least one processor calculates the LVEDP as a function of the cardiac time intervals, and an electrocardiogram (ECG).

In some embodiments, the method further includes calculating the contractility feature based on a cardiac waveform and an electrocardiogram (ECG), wherein the contractility feature comprises a derivative of the cardiac waveform data.

In some embodiments, the explicit or implicit time features and waveform features of the one or more signals comprises cardiac vibrations (seismic and or sound waves), and the method further comprising collecting a cardiac vibration waveform.

In some embodiments, the method further includes correcting the calculating of the LVEDP for valvular diseases based on the cardiac vibration waveform.

In some embodiments, the method further includes monitoring, by the at least one processor, the contractility feature by an imaging modality including at least one cardiac magnetic resonance imaging or an echocardiogram.

In some embodiments, the monitoring of the contractility feature is based on an electrocardiogram (ECG) and on at least one cardiac waveform data of: impedance cardiography (ICG), phonocardiography (PCG), photoplethysmography (PPG), seismocardiography (SCG), ballistocardiography (BCG), gyrocardiography (GCG), or echocardiography (echo).

In some embodiments, the monitoring the contractility feature is based on at least one of calculating a surrogate ejection fraction (EF) from non-invasively measured cardiac time intervals or a cardiac waveform.

In some embodiments, an apparatus for approximation of left ventricular end diastolic pressure (LVEDP) or an pulmonary capillary wedge pressure (PCWP), includes a non-invasive cardiac waveform sensor, at least one of a non-invasive electrocardiogram (ECG) sensor, and one or more processors coupled to the non-invasive cardiac waveform sensor, the at least one non-invasive ECG sensor, and a memory, where the memory holds computer instructions that when executed by the one or more processors cause the apparatus to perform certain operations. The operations can include receiving cardiac waveform data (ICG, PCG, PPG, SCG, BCG, or GCG) from the non-invasive cardiac waveform sensor configured for coupling to a patient, receiving electrocardiogram (ECG) data via the non-invasive ECG sensor, determining at least one of a pre-ejection period (PEP) or an isovolumic contraction time (IVCT), based on simultaneous portions of cardiac waveform data and at least the ECG data from the non-invasive cardiac waveform sensor, calculating an LVEDP or a PCWP based on a contractility feature and at least one of the cardiac time intervals, and encoding the LVEDP or the PCWP as digital data for at least one of storage, transmission, or human-comprehensible output.

In some embodiments, the memory holds instructions for calculating the contractility feature comprising cardiac time intervals based on the cardiac waveform data.

In some embodiments, the memory holds instructions for calculating the contractility feature based on at least one of: electrocardiogram (ECG), impedance cardiography (ICG), phonocardiography (PCG), photoplethysmography (PPG), seismocardiography (SCG), ballistocardiography (BCG), gyrocardiography (GCG), or echocardiography (echo).

In some embodiments, the memory holds instructions for calculating the contractility feature based on at least one of: calculating a surrogate ejection fraction (EF) from non-invasively measured cardiac waveform.

In some embodiments, an apparatus for approximation of left ventricular end diastolic pressure (LVEDP) includes a non-invasive cardiac waveform sensor, at least one of a non-invasive electrocardiogram (ECG) sensor or heart vibration waveform sensor, and at least one processor coupled to the non-invasive cardiac waveform sensor, the at least one of the non-invasive ECG sensor or heart vibration waveform sensor, and a memory, where the memory holds computer instructions that when executed by the at least one processor cause the apparatus to perform certain operations. Such operations can include receiving cardiac waveform data from a non-invasive sensor coupled to a patient and at least one of electrocardiogram (ECG) data or heart vibration waveform data, synchronizing, the cardiac waveform data and the at least one of electrocardiogram (ECG) data or heart vibration waveform data, and calculating an LVEDP based on time features and waveform features of the cardiac waveform data and the at least one of electrocardiogram (ECG) data or heart vibration waveform data. A heart vibration waveform sensor can generally be any type of sensor that can generally substitute for an ECG sensor within contemplation of the embodiments and can provide the heart vibration waveform data.

In some embodiments, the memory holds instructions for encoding the LVEDP as digital data for at least one of storage, transmission, or human-comprehensible output.

In some embodiments, the memory holds instructions for determining, at least one of a pre-ejection period (PEP) or an isovolumic contraction time (IVCT), based on simultaneous portions of the cardiac waveform data and at least one of the ECG data or the heart vibration waveform data.

In some embodiments, the memory holds instructions for collecting a cardiac waveform. In some embodiments, the memory holds instructions for correcting the calculating of the LVEDP for valvular diseases based on the cardiac waveform.

In some embodiments, calculating the LVEDP is based on the time features and waveform features of the cardiac waveform data and the at least one of electrocardiogram (ECG) data or heart vibration waveform data is based on or supplemented with at least one of: calculating a surrogate ejection fraction (EF) from non-invasively measured cardiac waveform.

In some embodiments, the calculating the LVEDP based on the time features and waveform features of the cardiac waveform data and the at least one of electrocardiogram (ECG) data or heart vibration waveform data that is based on or supplemented with at least one of a noninvasively obtained cardiac waveform or biological information.

### BRIEF DESCRIPTION OF THE DRAWINGS

FIG. 1A illustrates a system for the extraction, identification, marking and display of the heart valve signals in accordance with one embodiment;

FIGS. 1B and 1C illustrate cardio pulmonary signal capture at the chest in accordance with various embodiments;

FIG. 2 is a flowchart of a method practiced by the system in accordance with one embodiment;

FIG. 3 illustrates multichannel signals captured from the sensor array on the chest shown in accordance with one embodiment;

FIG. 4 illustrates a cardiac cycle in relation with Electrocardiogram, acoustic and accelerometer sensors of the system in accordance with one embodiment;

FIG. 5 illustrates a heart anatomy and schematic representation of the cardiopulmonary sounds in relation to electrocardiogram;

FIGS. 6A, 6B, and 6C illustrate a method and a cardiac time interval measurement in accordance with one embodiment;

FIGS. 7A and 7B illustrate the marking of vibration objects or each valve into individual streams in accordance with one embodiment;

FIGS. 8A, 8B, and 8C illustrate the comparison of M1, T1, A2, and P2 timings and comparison of time calculations using different energy thresholds in accordance with one embodiment;

FIG. 9 is a flowchart of a method practiced by the system in accordance with an embodiment;

FIG. 10 is an illustration of a dictionary of signal-atoms (on a left side) and a weight matrix/loadings (on a right side) given by a sparse coding source separation algorithm in accordance with an embodiment;

FIG. 11 is an illustration of a sparse projection of an activation pattern including PCA projection followed by subspace rotation in accordance with an embodiment;

FIG. 12 is an illustration of a system based on machine learning (example, deep learning) that learns to estimate LVEDP or PCWP;

FIG. 13 is an example of data labels (pronounced dots or green stars if shown in color) for Aortic valve opening given by an automatic labeling algorithm applied to Hemotag single channel data in accordance with an embodiment.

FIG. 14 illustrates the LV pressure-volume loops with cardiac valve events;

FIG. 15 illustrates a cardiac cycle in relation with various cardiac time intervals (CTIs);

FIG. 16 illustrates a system for the extraction, identification, marking and display of the noninvasive cardiohemic signals in accordance with one embodiment;

FIG. 17 is a flowchart of a telehealth Remote Patient Monitoring (RPM) System in accordance with one embodiment; and

FIG. 18 illustrates the marking of vibration objects in accordance with one embodiment.

### DETAILED DESCRIPTION

The exemplary embodiments may be further understood with reference to the following description and the appended drawings, wherein like elements are referred to with the same reference numerals. The exemplary embodiments describe a system and method of marking the cardiac time intervals and display of the heart valve signals. Specifically, psychoacoustics are considered in identifying the separated cardiac vibration signals captured through the transducers. The system, the psychoacoustics, and a related method will be discussed in further detail below.

The exemplary embodiments provide a novel approach for small, portable, robust, fast and configurable source separation based software with transducer hardware. The use of a vibration signal pattern and novel psychoacoustics help bypass conventional issues faced by linear time invariant systems. Clinical indices of myocardial contractility can be categorized as follows based on pressure measurements (such as dP/dtmax), volume and dimension (such as stroke volume and ejection fraction) and systolic time intervals (such as pre-ejection period, left ventricular ejection time and isovolumic contraction time). dP/dtmax is the gold standard of measurement of myocardial contractility. Some of the cardiac time intervals can include Left Ventricular Systolic Time (LVST), Left Ventricular Diastolic Time (LVDT), Pre-atrial Diastolic Filling Time (PADT), Accelerated Atrial Filling Time (AAFT), QS1 (Electromechanical activation time), QS2, Pre-Ejection Period (PEP), Right Ventricular Systolic Time (RVST), Left Atrial Systolic Time (LAST), Right Atrial Systolic Time (RAST), Right Ventricular Ejection Fraction (RVEF), Right Ventricular Diastolic Time (RVDT), Left Atrial Diastolic Time (LADT), Right Atrial Diastolic Time (RADT), Systolic Time Interval (PEP/LVST).

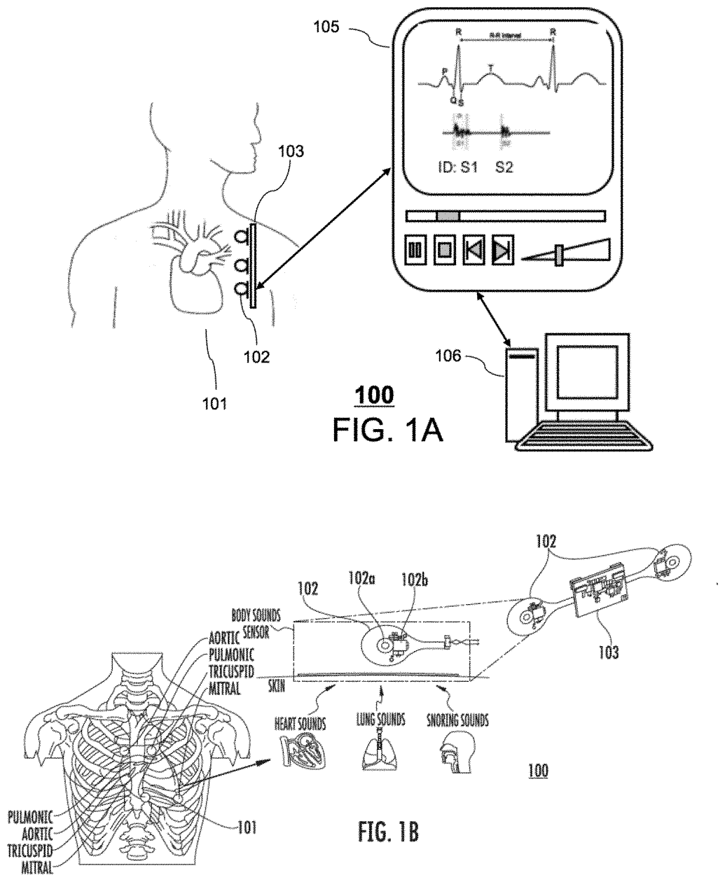

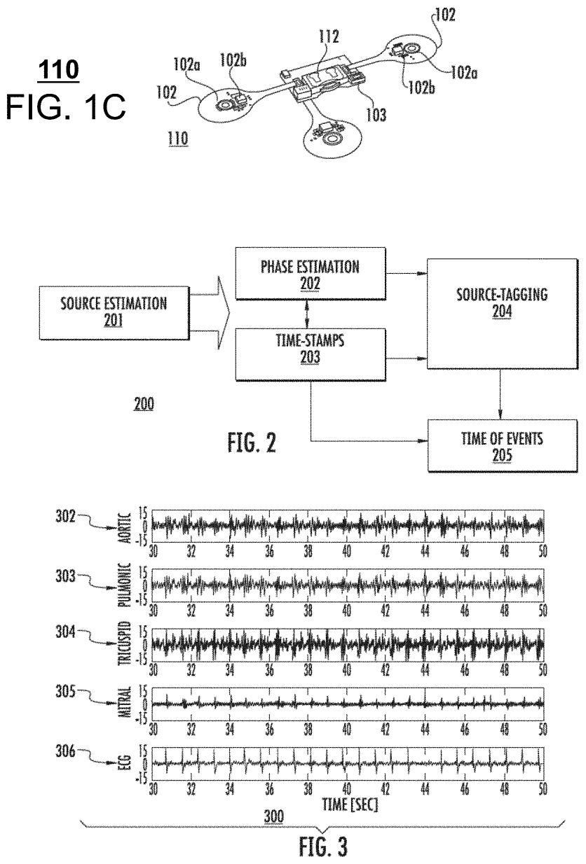

The exemplary embodiments of the system and method proposed here are shown in FIGS. 1A, 1B, and 1C. System 100 shown in FIGS. 1A and 1B is an embedded platform which can be any smart processing platform with digital signal processing capabilities, application processor, data storage, display, input modality like touch-screen or keypad, microphones, speaker, Bluetooth, and connection to the internet via WAN, Wi-Fi, Ethernet or USB. This embodies custom embedded hardware, smartphone, iPad-like and iPod-like devices. Area 101 in FIGS. 1A and 1B is the auditory scene at the chest locations. Array 102 in FIGS. 1A and 1B is the transducer array used to capture the heart signal(s). In some embodiments, the transducer array includes a pad that includes a vibration sensor such as a vibration sensor 102b and an electrode 102a for an ECG sensor. In some embodiments, the transducer array can include a single pad, two pads as shown in FIG. 1B or more than two pads as shown in FIG. 1C. In the particular embodiment of FIG. 1C, a transducer array 110 includes three pads (102) where each pad includes the vibration sensor 102b and the ECG electronic 102a. Other embodiments can include three or more pads where each pad would have at least a vibration sensor and optionally an electrode for the ECG sensor. Hardware 103 in FIGS. 1A-C is the wearable microprocessor hardware with digital signal processing capabilities, application processor, Analog to digital frontend, data storage, input modality like buttons, and wireless connection via Bluetooth, Bluetooth low energy, near field communication transceiver, Wi-Fi, Ethernet or USB.

Processor 112 shown in FIG. 1C comprises of the signal processing module on the wearable device that captures synchronized sensor data from the transducer array 102. The processor 112 is configured to save the synchronized sensor data to memory and communicate it with the system 100 for data transfer. Module 105 in FIG. 1A is the module that calculates vital sign from the input sensor stream coming from hardware 103 for the Heart rate, breathing rate, EKG signal, skin temperature, and associated vitals. The hardware 103 can optionally encrypt the raw sensor data for transmission to the cloud computing module 106. It can also communicate with a dashboard on module 105 or 106 for data exchange, login, alerts, notifications, display of processed data. Computing device 106 in FIG. 1A is the cloud module that processes the individual streams for eventual source separation. In some embodiments, the system 100 could include a connected display or other modality of display or presentation device. In some embodiments the system 100 allows a user to visually see the individual streams and information of the different cardiopulmonary signals.

The transducer array 102 can include multiple sensor transducers that capture the composite signal that includes the electrocardiogram signals, heart sounds, lung sounds and snoring sounds for example. The module 103 can be in the form of wearable hardware that synchronously collects the signals across the transducers and is responsible for the analog to digital conversion, storage and transmission to a portable unit 104. Note that the embodiments herein are not limited to processing the individual streams for source separation, identification and marking of the heart valve signals at the cloud computing module 106 only. Given sufficient processing power, the aforementioned processing can occur at the microprocessor hardware module 103, at the module 105, or at the cloud-computing module 106, or such processing can be distributed among such modules 103, 105, or 106.

The exemplary embodiments of the system and method proposed here for the source identification of the cardiopulmonary signals 200 are shown in FIG. 2. Block 201 indicates the separation of sources from the composite signals. Block 202 represents the phase estimation between the separated sources at each of the sensor position. Block 203 represents calculating the time stamps of individual sources at each heartbeat with respect to the synchronized EKG signal and the other sensor or sensors. Block 204 represents the source identification module responsible for tagging each of the separated source in individual heart beats to be one of the heart valve event, namely Mitral valve closing and opening, Tricuspid valve closing and opening, Aortic valve opening and closing, and the Pulmonic valve opening and closing. Block 205 represents the time marking module to estimate the time of occurrence of the above mentioned valve events with respect to the start of the EKG signal.

The exemplary embodiments of the system and method proposed here for the source identification of the cardiopulmonary signals from the composite signal 300 are shown in FIG. 3. Area(s) 101 in FIG. 1B indicate the locations at which the composite heart signal can be captured. A vibration signal 302 as charted on the first line in FIG. 3 represents a signal captured at the aortic auscultation location. A vibration signal 303 shows the vibration signal captured at the pulmonic auscultation location. A vibration signal 304 shows the vibration signal captured at the tricuspid auscultation location. A vibration signal 305 represents a vibration signal captured at the mitral auscultation location. The last or bottom line in FIG. 3 represents an electrocardiogram signal 306 captured. In some embodiments, note that the number of sensors used (such as in the sensor array 102 of FIG. 1), are less than the number of vibration sources. For example, 3 sensors can be used to ultimately extract signals for 4 (or more) vibration sources; or 2 sensors can be used to ultimately extract signals for 3 or 4 (or more) vibration sources; or 1 sensor can be used to ultimately extract signals for 2, or 3, or 4 (or more) vibration sources.

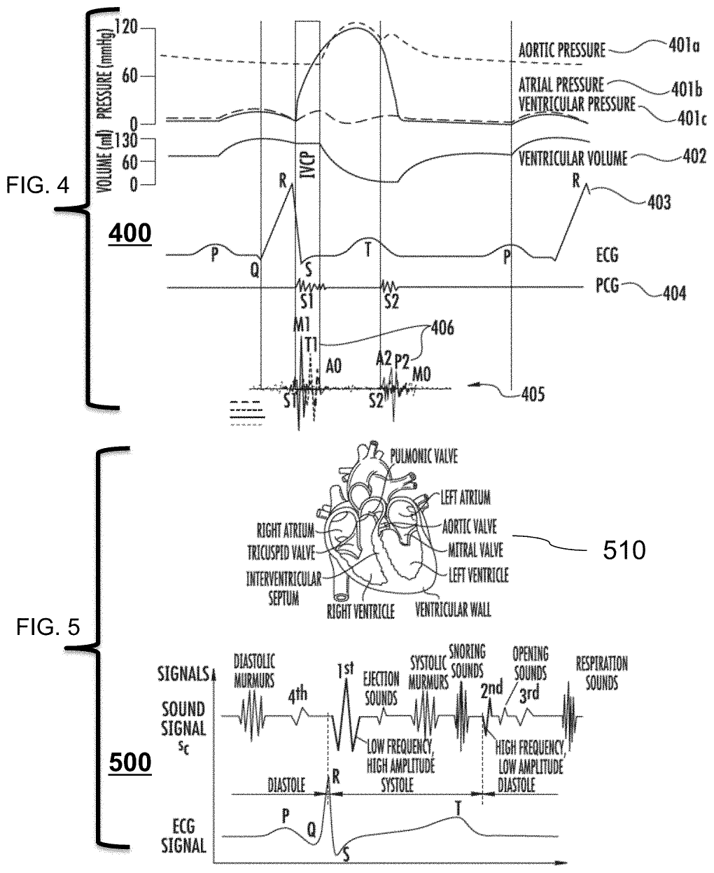

The exemplary embodiments of the system and method proposed here draw inspirations from biology with respect to the cardiac cycle in-relation with electrocardiogram and accelerometer transducer captured cardiac signal. A timeline chart 400 in FIG. 4 shows a cardiac cycle. Lines or signals 401a, 401b, and 401c represent or indicate the pressure changes during a cardiac cycle for aortic pressure (401a), atrial pressure (401b) and ventricular pressure (401c) measured in measured in millimeters of mercury (mmHg). Line or signal 402 represents or indicates the volume changes during a cardiac cycle in milliliters (ml). Line or signal 403 represents or indicates the electrical changes during a cardiac cycle captured by an electrocardiogram. Line or signal 404 represents or indicates the acoustic changes during a cardiac cycle captured by an acoustic sensor such as a phonocardiogram or PCG. S1 represents the first heart sound or the “lub” sound and the S2 represents the second heart sound or “dub” sound. Line or signal 405 represents or indicates the vibration changes during a cardiac cycle captured by an accelerometer transducer at the location of the device. Pattern 406 in FIG. 4 indicates the different valve opening and closing seen in line or signal 405 as captured by the accelerometer sensor or sensors. More specifically, a closer inspection of the pattern 406 reveals the closing of the mitral valve (M1) and tricuspid valve (T1) during the S1 or first heart sound and the closing of the aortic valve (A2) and pulmonary valve (P2).

FIG. 5 goes on to further show a representation 510 of the human heart relevant for the generation of the sounds and corresponding graph 500 representing the sounds belonging to coronary artery, murmurs, first sound, second sound, third sound, fourth sound, ejection sounds, opening sounds, respiratory sound, breathing, and snoring during individual heart beats, with respect to the electrocardiogram signal.

The exemplary embodiments of the system and method proposed here provide a source marking algorithm for the vibrations from the cardiohemic system. In some embodiments, the system next uses PCA to determine which source is associated with which event (e.g., Mitral closing & opening, Tricuspid closing & opening, Aortic opening & closing, Pulmonic opening and closing). The following describes the architecture for automatic source tagging and timing of valvular events. One way to identify which events are relevant to a source is by manually tagging the sources against the synchronized EKG signal and taking advantage of the timings relative to a QRS wave (identification of the S1 and S2 sounds using the EKG signal as the reference has been widely researched in studies). Another approach is an automatic tagging algorithm. The tagging is composed of a classifier preceded by a feature extraction algorithm. For the timing, the system exploits the computations of one of the feature extraction algorithms to obtain an energy contour from which the time location of a given event can be inferred. Because the embodiments here build upon having the ability to capture the signal at different locations simultaneously, to the proposed system exploits the relations among channels to extract additional information about the sources. Likewise, since some source separation algorithms where channels relations are associated with location, the system can leverage on the intrinsic relations among the channels to extract relevant information that helps the system distinguish among the events. In some embodiments, the system hypothesizes that phase information between channels is relevant for distinguishing among cardiac events since valves are located at different positions within the heart. Perhaps, one of the most distinctive features of the cardiac events is their relative order of occurrence, which repeats periodically with each heartbeat. Time information extracted from the set of sources can be utilized to localize the occurrence of each source signal within the heart cycle. Therefore, the features proposed here are conceived to provide three aspects: 1) Spectral information, 2) Relations among channels, and 3) Relations among events in the form of relative times of occurrence.

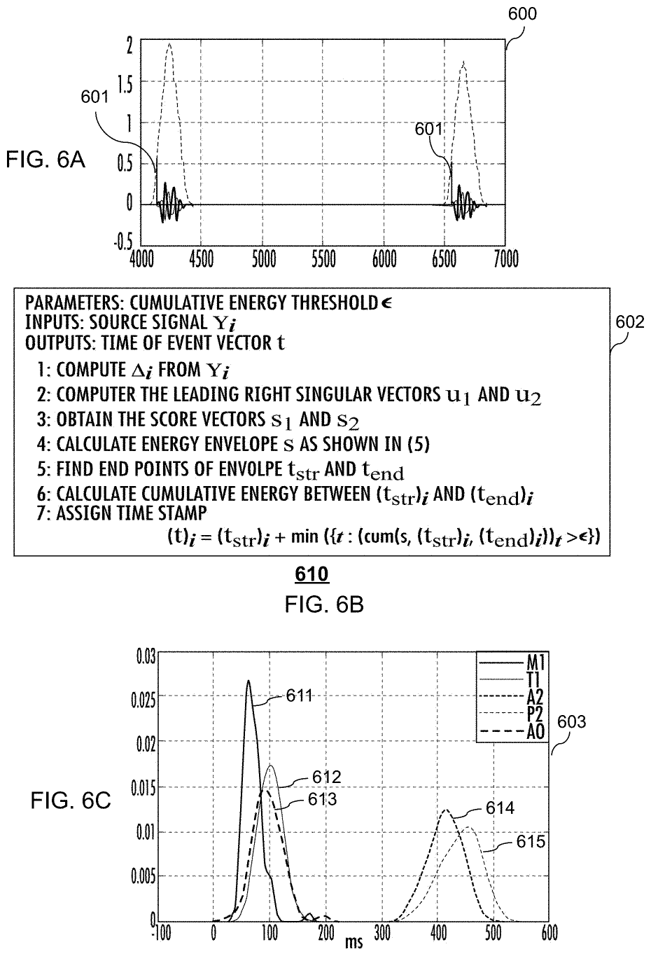

The automated timing is obtained from the separated sources. The embodiments can employ the eigenfilter approach described above to extract energy envelopes that can be easily detected and further processed to extract a time point. In this case, the system uses the two leading right singular vectors of the tap-delay matrix. It has been observed that, for a single channel, the first two right singular vectors of the tap-delay matrix contain oscillatory components with π/2 phase delay. This behavior can be extended to the two-channel case by noticing that the first half of the two leading singular vectors contain an oscillatory component of similar frequency with the above mentioned π/2 phase difference for channel 1, and that the same result applies to the second half for channel 2. From the above observation, we can consider the first 2 leading right singular vectors as a quadrature pair of eigenfilters. In other words, these filters have the same magnitude in frequency with a π/2 phase difference. The sum of instantaneous energies for the quadrature pair provides an energy envelope that, for the source signals, can be processed in a simple way to obtain time stamps on the occurrence of the events associated with the source. Let u1 and u2 be the two leading right singular vectors of Δi. Let s₁=Δᵢ u₁ and s₂=Δi u₂ be the score vectors. The energy envelope s can be calculated as (s)ₗ=(s₁)ₗ²+(s₂)ₗ². From the sparsity property of the heart sounds, it is possible to detect single heart beats from the energy contour s since the source signal is mostly zeroes followed by the oscillations related to the event at each heart beat. A simple marking procedure can be obtained by first detecting individual heartbeats and then processing the cumulative energy within a heartbeat to set a threshold that defines the marking point. Process 602 shown in the box 610 of FIG. 6B describes the procedure. A resulting time stamp (black vertical lines) 601 (in chart 600 of FIG. 6A) using the energy threshold can be marked. Notice that the endpoints of the Heart valve signal have been also detected as part of the procedure in determining the time stamps 601. The chart 600 shows the resulting markings using a cumulative energy to provide a threshold. In this case 1% of cumulative energy was selected to provide the threshold value. Chart 603 shows the time intervals found for the Mitral closing (611), Tricuspid closing (612), Aortic opening (613), Aortic closing (614) and Pulmonic closing (615).

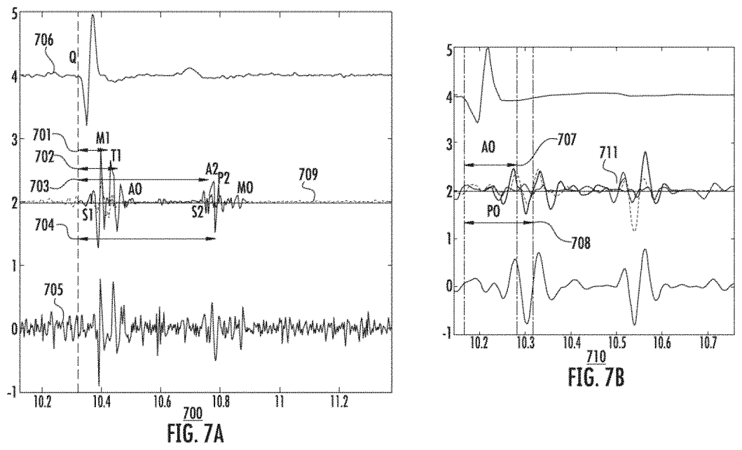

The exemplary embodiments of the system and method proposed here provide a source marking algorithm that allows from the explanation earlier for the marking of the Mitral valve closing (MC), Mitral valve opening (MO), Aortic valve opening (AO), Aortic valve closing (AC), Tricuspid valve closing (TC), Tricuspid valve opening (TO), Pulmonary valve closing (PC) and Pulmonary valve opening (PO) signals. The extracted individual valve vibration objects are aligned into a signal for each of the four valves across multiple heart beats. The chart 700 in FIG. 7A shows the source separation of heart valve opening and closing signals. Line 701 indicates the length or duration of the vibration signal for the Mitral valve closing (M1). Line 702 indicates the length or duration of the vibration signal for the Tricuspid valve closing (T1). Line 703 indicates the length or duration of the vibration signal for the Aortic valve closing (A2). Line 704 indicates length or duration of the vibration signal for the Pulmonic valve closing (P2). Signal 705 indicates the composite vibration signal captured by a particular transducer. Signal 706 indicates the EKG signal captured by the system. Referring to chart 710 of FIG. 7B, the Line 707 indicates the length or duration of the vibration of the Aortic valve opening (AO). Line 708 indicates the length or duration of the vibration of the Pulmonic valve opening (PO). Further note that the lines or signals 709 in FIG. 7A or 711 in FIG. 7B are actually several separated superimposed signals representing the vibration signals from separate sources coming from the mitral valve, tricuspid valve, aortic valve, and pulmonary valve (using less than 4 vibration sensors to extract such separated signals in some embodiments.

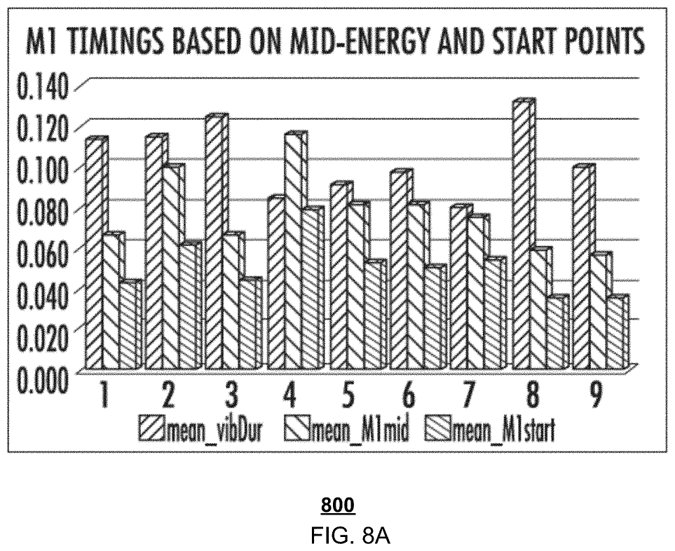

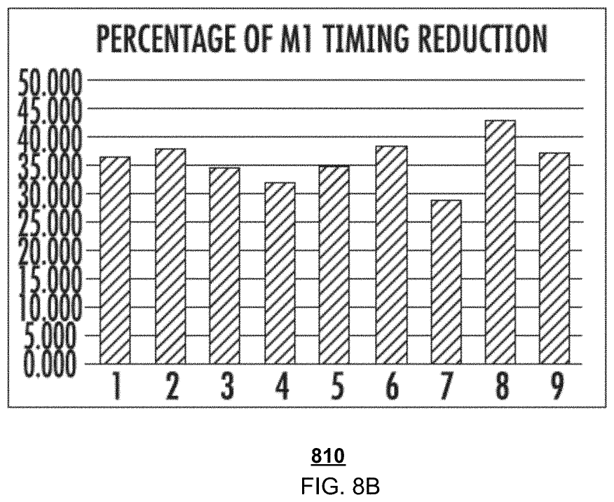

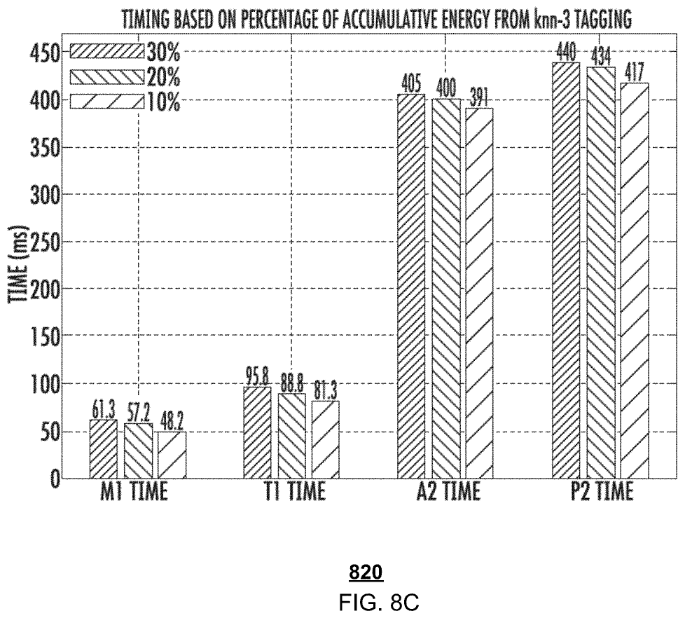

It was observed that peak of T1 timing distribution is close to that of AO. The reason is that the length of M1 and T1 Source Separation vibrations is longer than the length of AO Source Separation vibrations. So when the mid-point of accumulative energy is calculated, M1 and T1 timings are already shifted forward and don't represent the start of the vibration. Such a timing shift exists for AO but it's not as big as M1 and T1. To verify and compare, the following time information on some patients helps provide different approaches: Mean length of M1, T1 vibration, Mean start point of M1, T1 vibration, Mid-energy point is obtained from PCA algorithm. A shift back in timing of M1, T1, A2, P2 by reducing the 50% of accumulative energy to 30%, 20%, and 10%. The results are demonstrated in FIGS. 8A, 8B and 8C.

In the exemplary embodiments, a novel way of calculating the timing of the source separated individual heart vibration events from the composite vibration objects captured via multiple transducers is used to work on a single package, easy-to-use and portable device.

The exemplary embodiments develop a novel method of source timing, which in one embodiment using the Pulmonary and Aortic, and in addition possibly the Tricuspid and Mitral auscultation locations, lends the system capable of calculating the time intervals of individual valve events from the vibrations with respect to the electrocardiogram.

The exemplary embodiments develop a novel method of time interval calculation, which in one embodiment using the Pulmonary and Aortic, and in addition possibly the Tricuspid and Mitral auscultation locations, lends the system capable of marking the time of occurrence of the individual valve events with respect to the electrocardiogram. The novel method lends the system capable of measuring the cardiac time intervals.

The exemplary embodiments develop a novel method of providing time intervals of individual valve signals over time. The novel method allows for both short-term and long-term discrimination between signals. Short-term pertains to tracking individual stream when they are captured simultaneously as part of the composite signal. Long-term tracking pertains to tracking individual streams across multiple heart beats, tracking valve signals as they transition in and out during each cardiac cycle.

The exemplary embodiment of system and method described is the development on an embedded hardware system, the main elements required to capture body sounds are the sensor unit that captures the body sounds, digitization, and digital processing of the body sounds for noise reduction, filtering and amplification. Of course, more complicated embodiments using the techniques described herein can use visual sensors, endoscopy cameras, ultrasound sensors, MRI, CT, PET, EEG and other scanning methods alone or in combination such that the monitoring techniques enable improvement in terms of source separation or identification, and/or marking of events such as heart valve openings, brain spikes, contractions, or even peristaltic movements or vibrations. Although the focus of the embodiments herein are for non-invasive applications, the techniques are not limited to such non-invasive monitoring. The techniques ultimately enable diagnosticians to better identify or associate or correlate detected vibrations or signaling with specific biological events (such as heart valve openings and closings, brain spikes, fetal signals, or pre-natal contractions.)

The exemplary embodiments herein provide a method and system based on a technique to identify the separated cardiopulmonary signals, to extract information contained in vibration objects. In one embodiment, known under machine learning, auditory scene analysis, or spare coding approaches to the source separation problem. Data is obtained using a tri-axial accelerometer or multiple tri-axial accelerometers placed on different points of torso.

Examples of cardiac vibration objects are the first sound, the second sound, the third sound, the fourth sound, ejection sounds, opening sounds, murmurs, heart wall motions, coronary artery sounds, and valve sounds of the Mitral valve opening and closing, Aortic valve opening and closing, Pulmonary valve opening and closing, Tricuspid valve opening and closing. Examples of the pulmonary vibration objects are the respiratory lung sounds, breathing sounds, tracheobronchial sounds, vesicular sounds, Broncho vesicular sounds, snoring sounds. A portion of the energy produced by these vibrations lies in the infra-sound range, which falls in the inaudible and low sensitivity human hearing range. A portion of the energy produced by these vibrations falls in the audible hearing range. Accelerometer transducers placed on the chest capture these vibrations from both these ranges.

Source identification analysis in accordance with the methods described herein identify individual vibration objects described above from the source separated vibration signals. The individual vibration signals are identified to be from the mitral valve, aortic valve, tricuspid valve, the pulmonary valve, coronary artery, murmurs, third sound, fourth sound, respiratory sound, breathing, and snoring during individual heart beats. The identified signals are marked to indicate their start with respect to the start of the EKG.

The embodiments can include different source identification techniques specifically used for tagging the individual cardiopulmonary signals for application in a non-linear time variant system, such as Principal component analysis, Gabor filtering, Generalized Cross Correlation (GCC), Phase transform (PHAT), ROTH, SCOT and Band Filtering. Using 1) Spectral information, 2) Relations among channels, and 3) Relations among events in the form of relative times of occurrence.

The exemplary embodiments provide a novel approach for small, portable, robust, fast and configurable source separation based software with transducer hardware 103, 203. The use of the vibration signal pattern and novel psychoacoustics help bypass conventional issues faced by linear time invariant systems.

The following are the various types of heart disease that can be diagnosed and treated using the identifies signals, namely, Coronary artery disease, Heart murmurs and valve abnormalities, Heart failure, Heart rhythm abnormalities (arrhythmias), Vascular disease, congenital heart disease, and Risk factor modification. A physician can work with patients to perform a comprehensive evaluation and design a personalized plan of care aimed at keeping them healthy.

In another embodiment, the source can be identified by manually tagging them against the synchronized EKG signal and taking advantage of the timings relative to the QRS wave. This way, however is usually slow and time consuming and an automatic tagging algorithm is thus preferable. Since the different heart sounds comes from different locations in the heart, it is expected that each source will have a unique phase relation between the sensors that are located at fixed points during the data gathering phase. This phase relation can be made evident by using signal representation with a dictionary suited for highlighting frequency and phase relations with a greedy algorithm such as the matching pursuit. Our algorithm performs a Gabor analysis in the source separated signals using a finite Gabor Dictionary of fixed frequencies and with variable phase delay. The system finds the delay that minimizes the reconstruction error for each source and creates a group of features that will be incorporated into a decision-making and classification algorithm. The classification algorithm will combine the features extracted by the Gabor analysis with other features that comes from the PCA and cross correlation analysis that uses a set of manually tagged patient's tracks as training. The system is composed of three modules or stages: A suitable Gabor dictionary is created that can serve as the basis representation of the signals. An optimization algorithm aims to find the delay that will minimize the reconstruction error in a giving delay range. Collection and organization of all the features extracted from the second stage. For each one of the stages different techniques were tried in order to achieve the best results. The Gabor dictionary selected has the function:

$$G ( θ , n ) = {e}^{- \frac{1 {( n - θ )}^{2}}{2 {σ}^{2}}} cos ( 4 π \frac{n - θ}{f} )$$

In the equation θ is the delay, σ is the Gaussian decay of the atom and f is the frequency of operation of the Gabor. The system initially searched for the Gabor atoms that best represented the signal by doing a sweep in frequency and selecting those Gabor atoms that produced a larger weight matrix after the matching pursuit. The system was subsequently changed to a group of fixed frequency Gabor atoms exploiting the fact that for the M1, T1, A2 and P2 sounds vibration reside mainly in the range of 50-300 Hz and the Aortic opening vibration resides mainly in the low frequency range (from 0-50 Hz). The fixed Gabor approach sacrifices a little bit of reconstruction error in favor of a faster computation and easier analysis since the frequencies were fixed. The first optimization technique used was the, Gradient Descent algorithm: A first-order optimization algorithm that looks for the minimum of a given function by taking steps proportional to the negative of the gradient. The system is very dependent on the initial guess and number of minima of the system. A second approach was using the minimize function, which uses the Polack-Ribiere algorithm of conjugate gradients to compute search directions, and a line search using quadratic and cubic polynomial approximations. The system is also dependent on the initial value of the search but is more efficient in choosing the parameters to find the minimum. Finally, a brute force approach was implemented that sweeps the signal with different delays and then selects the one that resulted in a minimum function. In order to improve speed on this algorithm, a broad search is done first and then a refined search is done around the minimum point found by the broad search.

The exemplary embodiments of the system and method proposed here provide a source identification algorithm for the vibrations from the cardiopulmonary system. In order to find the time stamps for events such as Mitral closing & opening, Tricuspid closing & opening, Aortic opening & closing, Pulmonic opening and closing, we look at all the individual source separated signals 701 to 706 of the composite signal 707 and first tried to find the location of max peak in the SS signal for each source and then find delay between two channels. 708 shows the frequency spectrum of the source separated signals. Cross correlated vibrations in aortic and pulmonic channels for each interval for each source are calculated to find a consistent delay between two channels 709. Given the start of QRS and end point of each vibration, the vibration(s) within this interval is cross correlated with all vibrations in each source. This is done for both aortic and pulmonic channels. At the end, Principle Component Analysis (PCA) was applied to find the timing information and delay between two channels. PCA uses SS signal from each source in each channel to find the template which represents majority of the vibrations within that source. The template is then cross correlated with the whole source and max of PCA signal in each interval is found and compared with the start of QRS. In another implementation, In the second attempt, SS signals from two channels but the same source are fed into PCA to find the template. Then aortic template is used for both channels' cross correlation to identify the different vibrations into valve events, breathing sounds, and vibrations of the heart walls. To accurately estimate M1, T1, A2, P2, A0, P0 from the frequency signal captured by digital accelerometer on the wearable, several sub-frequencies are considered. Some are noted here for example: 0-30 Hz, 0-60 Hz, 30-150 hz, 30-250 Hz.

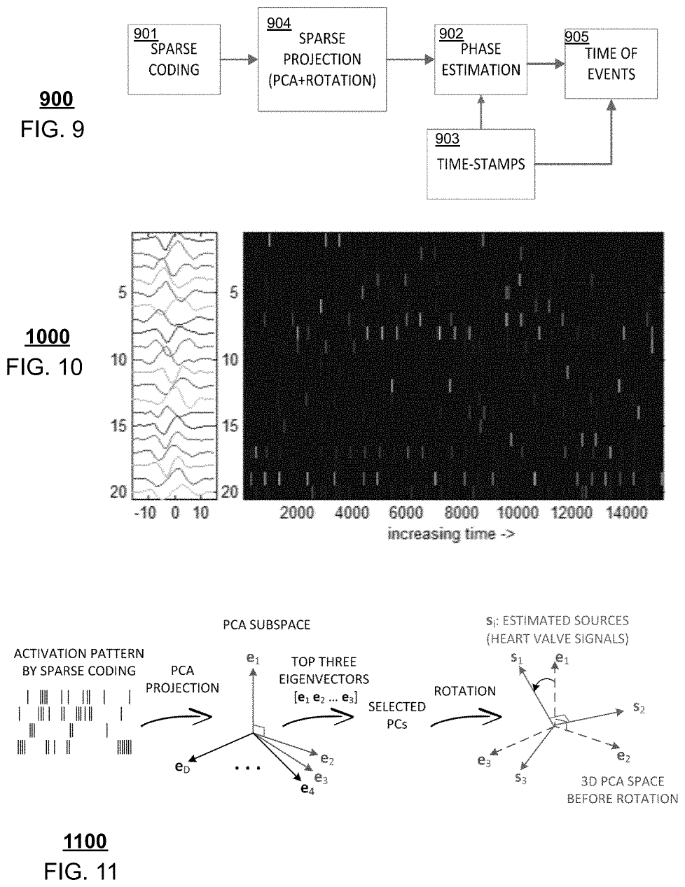

The exemplary embodiments of the system and method proposed here provide a source marking algorithm for the vibrations from the cardiopulmonary system. Next step is to use PCA to determine which source is associated with which event (Mitral closing & opening, Tricuspid closing & opening, Aortic opening & closing, Pulmonic opening and closing). We describe the architecture for automatic source tagging and timing of valvular events. One way to identify which events are relevant to a source is by manually tagging the sources against the synchronized EKG signal and taking advantage of the timings relative to the QRS wave (identification of the S1 and S2 sounds using the EKG signal as the reference hast been widely researched in studies. Another approach is an automatic tagging algorithm. The tagging is composed of a classifier preceded by a feature extraction algorithm. For the timing, we exploit the computations of one of the feature extraction algorithms to obtain an energy contour from which the time location of a given event can be inferred. Because our work builds upon having the ability to capture the signal at different locations simultaneously, we want to exploit the relations among channels to extract additional information about the sources. Likewise some source separation algorithms where channels relations are associated with location, we leverage on the intrinsic relations among the channels to extract relevant information that helps us distinguish among the events. We hypothesize that phase information between channels is relevant for distinguishing among cardiac events since valves are located at different position within the heart. Perhaps, one of the most distinctive features of the cardiac events is their relative order of occurrence, which repeats periodically with each heartbeat. Time information extracted from the set of sources can be utilized to localize the occurrence of each source signal within the heart cycle. Therefore, the features we propose here are conceived to provide three aspects: 1) Spectral information, 2) Relations among channels, and 3) Relations among events in the form of relative times of occurrence. We describe a feature extraction algorithm based on multichannel Gabor basis decomposition using matching pursuit, and a second algorithm that uses eigen-decomposition of a covariance matrix extracted from the source signals to obtain a cross correlation function between channels. For the gabor basis, consider the reconstruction of a 2-channel signal x (t). In this approach we want to extract a phase relation between the sensors which are located at standard aortic and pulmonic auscultation positions. As we mentioned above, we are interested on features that reflect spectral content as well as the channel interrelations. In the two channel case, each basis function is a pair of Gabor functions with equal frequency f and envelope width h and a phase difference θ. The tunable parameter θ can be swept across a range of values for which the matching pursuit reconstruction error can be obtained. The behavior of the reconstruction error for a source signal over different values of θ provides information about the phase different between the two channels for the particular source signal. In particular, the value of θ that attains the minimum error within the defined range of values is taken as the optimal channel delay, and the time average of the activation coefficients obtained by matching pursuit at the optimal θ provide and spectral characterization of the source. For the feature Extraction by Self-Similarity Template Based on PCA, we define a self-similarity using the concept of eigenfilter. Let yi the a two-channel source signal defined in (1), and let Yi its discretized version of size N×2. For filter of length Nw, we compute the tap-delay matrices Δ(j). The joint-channel eigenfilter correspond to the leading right-singular vector u of the tap-delay centered matrix Δi obtained by removing the mean of each column of Δi. The eigenfilter u can be split into the first and second channel components. The first channel component u(1) corresponds to the first Nw entries of u, and u(2) to the remaining entries going from index Nw+1 to last index 2Nw. The proposed feature is the cross-correlation function between u(1) and u(2). Note that this function contains the main frequency components of the source signals expressed in the time domain and the peak provides information about the channel relations. For different delays between the two channels the cross correlation peaks at different locations, accordingly. To extract relative time information contains cues for the classification of the cardiac events, we adopt a simple approach that uses the energy contours. The process consists of three basic steps: Compute energy contours for all source signals, Compute timings for each source signal, Compute features of source i by averaging the sum of the remainder sources centred at the timings. The energy of each source signal Yi is calculated using the leading, quadrature pair of right singular vectors of their corresponding tap-delay matrix. For classification, a set of training exemplars (sources) have been manually tagged with the respective events. Each source signal is then represented by the feature vectors described above. Test have been carried out on pooled covariance linear discriminant analysis, quadratic discriminant analysis, k-nearest neighbours and support vector machines both linear kernel and Gaussian kernel. The features described in the previous sections can be used in different manners for tagging, for example: Tagging using only Gabor-based features, Tagging using only self-similarity features, Tagging using only time-based features, Tagging using all Gabor-based, self-similarity, and time-based features. There are even sub cases of the situations considered above. For instance, the combination of Gabor and self-similarity features can be done on a single classifier or using ensembles. The first validation examples only address the cases in the bullet items. In addition to the classifier, preprocessing of the feature vectors is also performed. A centering vector x_{cent} is calculated using the mean over the training set Xₜᵣₐᵢₙ. This step is followed by a linear transformation based on PCA to produce a much smaller dimension feature vector. The preprocessing is summarized in Algorithm 1, FIG. 9. The choice of p is typically driven by the training data itself. The singular values of Xₜᵣₐᵢₙ can be used to decide the dimensionality of the transformed feature vectors after.

The exemplary embodiments of the system and method proposed here provide a source marking algorithm that allows from the explanation earlier for the marking of the Mitral valve closing (MC), Mitral valve opening (MO), Aortic valve opening (AO), Aortic valve closing (AC), Tricuspid valve closing (TC), Tricuspid valve opening (TO), Pulmonary valve closing (PC) and Pulmonary valve opening (PO) signals. The extracted individual valve vibration objects are aligned into a signal for each of the four valves across multiple heart beats. 800 in FIG. 8 show the source separation of heart valve opening and closing signals. 801 indicate the vibration signal for the Mitral valve closing. 802 indicate the vibration signal for the Tricuspid valve closing. 803 indicate the vibration signal for the Aortic valve closing. 804 indicate the vibration signal for the Pulmonic valve closing. 805 indicate the composite vibration signal captured by a particular transducer. 806 indicate the EKG signal captured by the system. 807 indicate the vibration of the Aortic valve opening. 808 indicate the vibration of the Pulmonic valve opening.

The exemplary embodiments of the system and method proposed here provide a source marking algorithm for the vibrations from the cardiopulmonary system, using information about the time of occurrence of the event. Automated finding of M1, T1, A2 & P2: After going through the timing plots of different patients, it was decided to first determine A2 and P2 timing and their corresponding sources and then find M1 and T1 from the rest of sources. Automatic A2 & P2 finding: 1) The number of zero-crossings is found for each SS source. Then the noisy source that is associated with the maximum zero-crossing is discarded. 2) Automated calculation of QRS onset points for all heartbeats in each source. 3) Automated calculation of beginning and ending points of all vibrations in each source. 4) Applying PCA approach on all sources that outputs the time difference between QRS onset and peak of PCA signal (timing vector) as well as delay between aortic and pulmonic channels. 5) Based on probability density estimation of each source's timing vector, strong peak(s) that correspond(s) to timing(s) more than 300 ms are accepted as A2 and P2 candidates and those sources that don't satisfy this condition are discarded. Also new candidate timings are sorted in ascending order. 6) Variation, length of samples more than 300 ms (L) and some other measurements are calculated from accepted timing vectors of accepted sources and are stored in a structure for further analysis. 7) If there is only one strong peak in probability density estimation plot whose timing is more than 300 ms, then a variable called “ind_both” is set to 1. If there are two or more strong peaks whose timing is more than 300 ms, if max peak's timing is over 300 ms, then “ind_both” variable is set to 1 but if max peak's timing is lower than 300 ms but another strong peak's timing is more than 300 ms, then “ind_both” is set to 0. If there are two or more sources whose “both_indic” variables are equal to 1, then “both_indic”=1. 8) In case of having no valid timing, A2 and P2 are set to zero. 9) In case of having one valid timing, if L>6 then it is marked as A2 and its corresponding source number is also saved. P2 is set to zero. 10) In case of having two valid timings:

if both_indic =1 ,

if L1 >= 6 samples, then A2 is set to first timing in sorted timing vector Else A2=0
if A2=0 and L2 >=6, then A2 is set to second timing in sorted timing vector Else P2=0
if A2 ~= 0 and L2 >=6, then P2 is set to second timing in sorted timing vector Else P2=0

```
if both_indic ~=1 ,
  if L1 >= 4 samples & V1<=800 then A2 is set to first timing in sorted timing vector Else A2=0
  if A2=0 and L2 >=4 & V1<=800 , then A2 is set to second timing in sorted timing vector Else
P2=0
  if A2 ~= 0 and L2 >=6 & V2<=800 then P2 is set to second timing in sorted timing vector Else
P2=0
```

| | |
|---|---|
| 11) | In case of having more than two valid timings, |

if both_indic =1
for loop: if L1 >=6 & V1<=800, then A2 is set to current timing in sorted timing vector; break

Else A2=0; End of for loop

```
  if A2=0, then P2 = 0;
  if A2~=0 & last timing in the sorted timing vector
```

if L >=6 & V1<=800, then P2 is set to last timing in the sorted timing vector

if A1~=0 & there are two timings after A1 timing,

```
  if V2<V1, then P2 is set to second timing after A2.
  Else P2 is set to first timing after A2.
```

if A1~=0 & there are more than two timings after A1 timing,

if L1>=6 & V1<=800 , then P2 is set.

```
if both_indic ~=1 ,
for loop: if L1 >= 8 samples & V1<=1000 then A2 is set to current timing in sorted timing vector
Else A2=0; End of for loop.
  if A2=0, then P2 = 0;
  if A2~=0
```

| | |
|---|---|
| for loop: | if L >=8 & V1<=1000, |
| | then P2 is set to current timing in the sorted timing vector; break |
| | Else P = 0; |

End of for loop
At the end A2 and P2 timings and their corresponding source numbers are saved in an excel
with a long with patient name.
Automatic M1 & T1 finding:

| | |
|---|---|
| 1) | The same previous steps from 1 to 7 are implemented in this phase |
| 8) | In case of having no valid timing, M1 and T1 are set to zero. |
| 9) | In case of having one valid timing, if L > 6 and timing is less than 120 ms, |

```
      then it is marked as M1 and its corresponding source number is also saved.
      P2 is set to zero.
```

| | |
|---|---|
| 10) | In case of having two valid timings, |

```
if both_indic =1 ,
  if L1 >=6, then M1 is set to first timing in the sorted timing vector Else M1 =0
  if M1 =0 & L2 >=6, then M1 is set to second timing in the sorted timing vector and T1=0
  if M1~=0 & L2>=6 and second timing is less than double the first timing
```

then T1 = second timing in the sorted timing vector Else T1 =0

if both_indic ~=1
L1>=4 & V1<=800, then M1 is set to first timing in the sorted timing vector Else M1 =0
If M1=0 & L2>=4 & V2<=800, then M1 = second timing in the sorted timing vector Else T1 =0
If M1~=0 & L2>=4 & V2<=800, then T1 = second timing in the sorted timing vector Else T1 =0

| | |
|---|---|
| 11) | In case of having more than two valid timings, |

if both_indic ~=1 ,
for loop: if L >=8 & V<=1000, then M1 is set to current timing in the sorted timing vector Else
M1 =0; break; end of for loop
if M1 =0, then T1 = 0;
for loop: if M1~=0 & L >=6 & V<=1000, then T1 is set to current timing in the sorted timing
vector; break, end of for loop
if M1~=0 & L2>=6 and second timing is less than double the first timing

then T1 = second timing in the sorted timing vector Else T1 =0

if both_indic =1
low_ var = Find sources with variations less than 15.
From these low_var sources, find those whose length are more than 9 samples(len_low_var).
If length of low_var >=2 & len_low_var >=2

```
  M1 is set to the timing corresponding to the lowest variation
  T1 is set to the timing corresponding to the second lowest variation
```

Else
for loop: If L >=5 & V<800, then M1 is set to the current timing in the sorted timing vector Else
M1=0
If M1 =0, then T1 =0
If M1~=0
for loop:
If there is only one timing after M1 & L >=5 & V <=800, then T1 = current timing in the sorted
timing vector; break
If there are two timings after M1,
if V1 < 300, then T1 = first timing after M1 in the sorted timing vector; break
Elseif V2<V1, then T1 = second timing after M1 in the sorted timing vector; break
Else, T1 = first timing after M1 in the sorted timing vector; break
If there are more than two timings after M1,
if V1 < 5, then T1 = current timing after M1 in the sorted timing vector; break
Elseif V2<V1, then T1 = current timing after M1 in the sorted timing vector; break
Else, T1 = current timing after M1 in the sorted timing vector; break
---
Automated finding of Aortic opening (AO):

| | |
|---|---|
| • | Track 1 with 2 channels 8 by 2 by N |
| • | M1, T1 timings and EKG times |
| • | Remove the noisy source using max zero-crossing. Data down to 7 by 2 by N |
| • | Sub-source markings including start and end of vibrations, envelope and max peak |
| • | Save to non-zero-source vector for both channels |
| • | Remove missing start and end points and too large ones by comparing between two |
| | channels as well as considering each source |
| • | Calculate PCA for two channels and all vibrations. Outputs are delay_vec, PCA signal and |
| | delay between two channels |

Begin AO detection

| | |
|---|---|
| • | Select above data for AO channel |
| • | Remove zero delay values |
| • | Remove delays between two channels that are greater than 50 ms and count < 4 |
| • | Remove positive delays between two channels and count <5 |
| • | Calculate ksdensity |
| • | Find max peak as well as other peaks that their distance together is more than 5 |
| | samples |
| • | Keep negative delays if delay is less than 150 ms |
| • | Calculate ksdensity of event_vec |
| • | Find more max peak and peaks > half of max peak |
| • | Find peaks > 10 ms and with 0.05 of max probability value |
| • | Save all the candidate sources with negative delay btw two channels |
| • | Select peaks that fall in range of M1 and M1 + 95 ms (if M1=T1=0 , in range 20 to 150 |
| | ms) |
| • | Sort all timings and save |
| [symbol] | Find AO |

First case: If there is only on candidate source:

| | |
|---|---|
| 1) | If delay variation < 500 but source variation > 1000, |
| 2) | remove timings > 200 ms, |
| 3) | If length of samples > 4, remove samples whose timings relative to median are > 60 ms |
| 4) | If empty timing vector: |

```
I. then get the original timing vector, calculate kdensity and find max and other peaks whose
probability is more than half of max's probability
II. If time diff btw AO candidate and max peak's timing < 10 ms,
      set max's peak timing as ref time, remove samples with diff more than 50 ms relative to
ref time and update the AO candidate and its variation.
III. If time diff btw AO candidate and max peak's timing > 10 ms,
    find a qualified peak and its corresponding timings that is closest to AO candidate, set that
peak's timing as ref time, remove samples with diff more than 50 ms relative to ref time and
update the AO candidate and its variation.
```

| | |
|---|---|
| 5) | If non-empty timing vector: |
| I. | calculate kdensity and find max and other peaks whose probability is more than half of |
| | max's probability. Find the one that is closest to candidate, set is as ref time, remove |
| | samples with diff more than 50 ms relative to ref time and update the AO candidate and |
| | its variation. |
| 6) | Check if var_delay < 500 && var_SRC < 900 && AO_candidate <= M1 + 96 |

Second case: If there are two candidate sources:
The following steps from the first case are implemented:

| | |
|---|---|
| • | 1, 2, 3 with 50 ms threshold, and 6 |
| • | After founding all valid AO timings from all candidate sources, the closet AO candidate |
| | to “ M1 + 30 ms” is selected. |

Third case: If there are more than two candidate sources:
The following steps from the first case are implemented:

| | |
|---|---|
| • | 1, 2, 3 with 90 ms threshold, 4 with 90 ms threshold, and 6 |
| • | After founding all valid AO timings from all candidate sources, the closet AO candidate |
| | to “ M1 + 30 ms” is selected. |

To improve the results in the exemplary embodiment we develop AO detection using new PCA approach such as: Mean removal, Min-Max removal, Different window length for correlation. Detailed analysis on delay calculation from cross-correlation template. Source tagging using PCA features and different classifiers on both tagged and untagged tracks for both High Pass and Low Pass. Defined two timing calculation algorithm 1) with threshold 2) without threshold and got the timing distributions for different test cases. To improve AO detection using new PCA algorithm, a few different tests were done such as: Removed the mean of each source's template (X matrix) before calculating covariance matrix. For some sources, cross-correlation template shows zero delay at a discontinued point although there is a delay between peaks of templates. Removed min-max of eigenvector before performing cross-correlation showing a continuous behavior with a delay. Then ran different SSs on the same patient to see if these observations are consistent. There are different scenarios when calculating the delay such as: Maximum peak is positive or negative, The Delay of Max peak is positive or negative. Our approach is to select positive maximum peak with the positive delay. The goal is to tag or label the sources with an event (HP: M1, T1, A2, P2, LP: AO). The cross-correlation signals generated by new PCA algorithm are used as features to train the classifiers and then tag sources of a test signal. Similar approach can be extended for the identification of other cardiopulmonary signals, like coronary artery sounds, snoring, murmurs, respiration sounds in one embodiment.

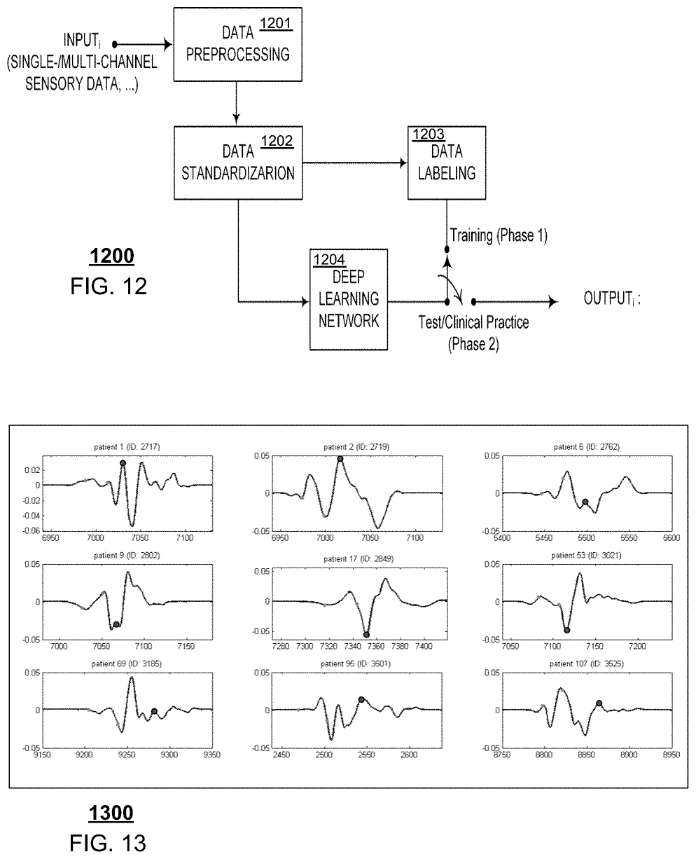

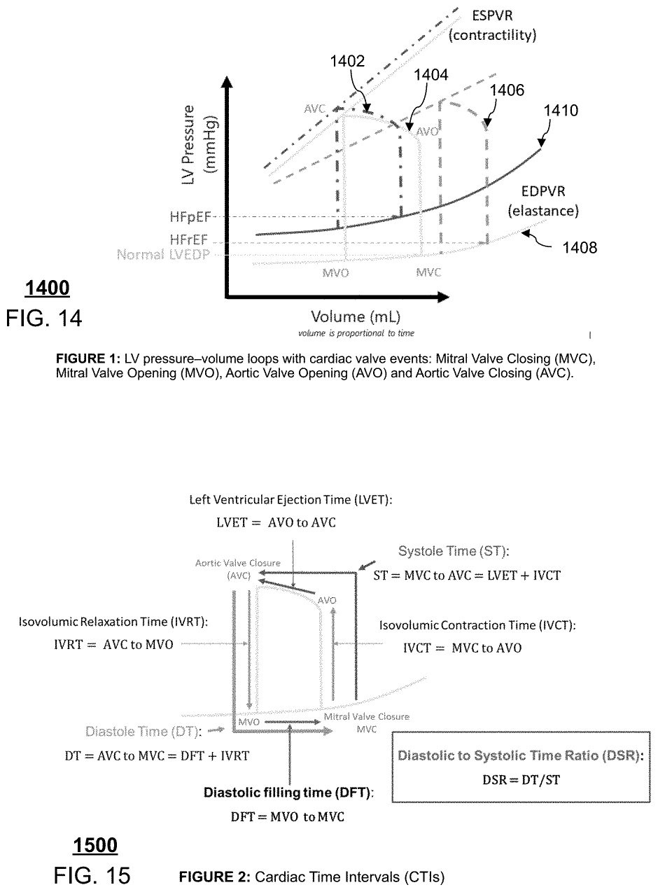

Referring to FIGS. 9-11, The exemplary embodiments of the system and method proposed here provide a source marking algorithm for vibrations from the cardiohemic system. In some embodiments, the system includes a sparse projection of a deficient activation pattern given by an underdetermined source separation algorithm (see FIG. 9). The activation patterns (see FIG. 10) are projected to a possibly lower dimensional space of reconstructed sources. The projection consists of an unsupervised principal component analysis (PCA) metric projection, followed by a regularization step to make the projection loadings sparser. The regularization is performed by an orthogonal rotation of PCA subspace, and helps to better understand the rotated space which is now aligned with the space of valvular events (see FIG. 11). Referring now to FIGS. 12 and 13, diagnosis of cardiovascular diseases, including heart failure, are currently based on medical experts' interpretation of patient's physical examination and medical records using a well-developed flow-chart, comparing patient's conditions with a typical taxonomy of known conditions. However, this diagnostic procedure is not always accessible, and may be expensive or inefficient—as it is error-prone. An alternative approach to access appropriate medical diagnostic services is to use artificial intelligence and design rule-based expert systems or graphical models to consistently follow the same diagnostic procedure performed by a physician. However, designing a system to imitate the reasoning processes of human experts could be very challenging, as it requires significant rule extraction and feature engineering.

An alternative to artificial intelligence is deep learning that has recently produced considerable breakthroughs proven to be very efficient for image and signal analysis, like image classification and segmentation, speech recognition, language translation, and structured data analysis. Deep learning is based on deep neural networks with hierarchical intermediate layers of artificial neurons, which can progressively extract increasingly complex features. By learning very complicated input-output relationships, deep learning can outperform systems that are based on manual feature extraction, like rule-based expert systems.

A prerequisite for using deep learning is that there must be a thousand to millions of well-labeled data points (pairs of input-output) to train the deep network. With the new advances in cardiovascular technologies, which are able to capture large quantities of patients' data, this prerequisite is met. A recent survey (by Bizopoulos, P. and Koutsouris, D., 2018. Deep Learning in Cardiology. IEEE reviews in biomedical engineering, 12, pp. 168-193) summarizes the applications of deep learning in cardiology, where it is applied to patients' structured data, and signal and image modalities in cardiology of heart and vessel structures.

Despite of superior performance of deep learning for some cardiovascular applications, their use as a diagnostic black box prevents them to integrate in the clinical practices (as reported by Miotto, R., Wang, F., Wang, S., Jiang, X. and Dudley, J. T., 2017. Deep learning for healthcare: review, opportunities and challenges. Briefings in bioinformatics, 19(6), pp. 1236-1246).

We introduce a system (see FIG. 12) based on deep learning (DL) networks, which is applicable to any sensory database, image, motion image/video, single or multichannel sensor signals (from sensors such as ECG, accelerometers, gyroscopes, acoustic microphones, micro-electro-mechanical systems—MEMS, microwave, radar, radio-frequency, doppler, or Near Field Communication—NFC) and structured data and after proper training with pairs of cardiovascular input-output, can produce absolute markers, like heart valvular events (e.g., Mitral closing & opening, Tricuspid closing & opening, Aortic closing & opening, Pulmonic closing & opening), ejection fraction (EF), global longitudinal strain (GLS), left atrial volume index, different cardiac time intervals (CTI's), mean Pulmonary Artery pressures (mPAP), systolic Pulmonary Artery pressures (sPAP), and diastolic Pulmonary Artery pressures (dPAP), Pulmonary Wedge Capillary Pressure (PCWP), Cardiac Output and stroke volume. This is a totally different perspective of using deep learning, because we train the system with absolute markers so the immediate outputs of the deep learning after training are absolute markers. This is consistent with the concept of evidence-based medicine and partially satisfies the ethical and legal issues of using machine learning and artificial intelligence methods in clinical practices, which is currently a big challenge for their practical application (as discussed by Lee, E. J., Kim, Y. H., Kim, N. and Kang, D. W., 2017. Deep into the brain: artificial intelligence in stroke imaging. Journal of stroke, 19(3), p. 277).

The embodiment of the system and method proposed here includes a preprocessing step (noise removal, . . . ), a data standardization that brings the preprocessed data to a common format (normalization, scaling, windowing, . . . ). It also includes a manual or automatic data labeling step to extract the desired outputs (labels) for each particular input (FIG. 2, shows one such embodiment of the inputs, namely, the Aortic valve openings). On training session (Phase 1) the deep learning module learns from pairs of input/output,. At the end of training session the system is ready for test and clinical application (Phase 2).

The embodiment of the system and method proposed here is applicable for longitudinal monitoring of absolute markers over time, when the times series of markers provided over hours, days or weeks indicates if the patient's symptoms is improving/worsening.

The embodiment of the system and method proposed here can be used in a multidisciplinary framework to generate combined outpatient markers, like acute decompensated heart failure, heart failure with preserved ejection fraction (HFpEF) for diastolic dysfunction, heart failure with reduced ejection fraction (HFrEF) for systolic dysfunction pulmonary hypertension, different grades of diastolic dysfunction I, II, III, different grades of Mitral Valve Regurgitation (mild, moderate, moderately severe, or severe), different grades of Mitral Valve Stenosis (mild, moderate, or severe), different grades of Aortic Regurgitation (mild, moderate, moderately severe, or severe), different grades of Aortic Stenosis (mild, moderate, or severe), congestion in the lungs, Coronary Artery Disease, Lung Disease, Ischemic Cardiomyopathy, Left Ventricular Hypertrophy, Sleep Apnea, Angina, and Myocardial Infraction.

The deep learning core of the embodiment of the system and method proposed here, can be deep feed forward (DFF), deep belief network (DBN), deep convolutional network (DCN), deep convolutional inverse graphics network (DCIGN), Deep Boltzmann Machine (DBM), or any other deep structure.

The present application relates to systems and methods for noninvasive evaluation and monitoring of elevated left ventricular end-diastolic pressure (LVEDP) or pulmonary capillary wedge pressure (PCWP) using at rest assessment of hemodynamic performance, based on quantitative measurements of heart and lung related parameters and cardiac events for diagnostic and therapeutic purposes, that can include one or a plurality of signals from one or a plurality of noninvasive sensors or transducers that measure one or a plurality of physiological effects that are correlated with cardiopulmonary functions; transmission of the data to a computing device and analysis software; a trained algorithm used to process the data to determine the state or condition of elevated LVEDP or PCWP; and an output indicative of the state or condition of the analysis.

Heart catheterization is currently the gold standard for assessing pulmonary hemodynamics and diagnosing serious conditions such as pulmonary hypertension (PH) because it confirms pulmonary pressures and valvular, myocardial, or congenital causes (if present) in an invasive manner. Although generally considered safe, heart catheterization is an invasive procedure associated with occasional risks including air embolism, arterial puncture, bleeding, brachial plexus/phrenic nerve injury, lung puncture (pneumothorax), pulmonary infarction, right bundle branch block, and tachycardia. Complications can range from minor discomfort at the site of catheterization, to major ones such as acute renal failure, stroke, and death. Therefore, signs and symptoms are still the most common method used at point-of-care, and options for the management of these patients remain crude and limited. Even with all the advances in medical science, there is no simple on-the-spot method for assessing a patient's pulmonary hemodynamics. Lead I ECGs are good for detecting arrhythmias and scales to detect weight gain. The potential role of biomarkers in screening and diagnosing PH has been the subject of increasing interest over the last decade. Brain natriuretic peptide (BNP) and N-terminal BNP (NT-proBNP) are two biological substances found in the blood that have been studied as a screening test in patients at risk for PH. Other biomarkers currently under investigation include atrial natriuretic peptide, endothelin-1, uric acid, troponin T, nitric oxide, asymmetric dimethylarginine, cyclic guanosine monophosphate, D-dimer, and serotonin. Blood test may not be available at all facilities, and having blood drawn with a needle caries its own risks, including excessive bleeding, fainting, hematoma, and infection. Furthermore, common over-the-counter doses of biotin supplements interfere with assays for NT-proBNP, and usually patients are required to stop biotin consumption for at least 72 hours prior to the collection of a blood sample.

Pulmonary Capillary Wedge Pressure (PCWP) is frequently used as an estimate of LVEDP. It is measured invasively by inserting a balloon-tipped, multi-lumen catheter (Swan-Ganz catheter) into a central vein and advancing the catheter into a branch of the pulmonary artery. Pulmonary hypertension related to left heart disease (LHD) accounts for 65-80% of PH cases. Research has shown that reliance on the PCWP can lead to misclassification of PH, resulting in harmful or costly use of pulmonary vasodilators for patients with LHD, and limiting the ability to detect beneficial drug effects. Systolic dysfunction in patients with PH and left ventricular (LV) heart failure (HF) with reduced ejection fraction (HFrEF) is typically easier to detect, while PH associated with preserved ejection fraction (HFpEF) is significantly more challenging. The gold standard for diagnosing LV diastolic function requires an invasive assessment of the pressure-volume relationship. It has been shown that LV stiffness is a reliable characteristic in patients with HFpEF.

An alternative to invasive hemodynamics assessment is an echocardiographic assessment for HFpEF, including the estimation of LVEDP by early mitral filling velocity (E) to early diastolic mitral annular velocity (E′) ratio, assessment of left atrial size, and deceleration time. However, not only is an echocardiogram expensive ($1000-$3000) and time consuming (20-60 minutes), but research indicates that some of the measures may not be reliable predictors of left ventricular filling pressure, e.g., in severe mitral regurgitation. PH is a crucial clinical and pathophysiologic feature of HFpEF.

Given the risk, cost and availability of cardiac catheterizations, impracticality of regular echocardiography assessment and lack of validated biomarkers for diagnosing and managing PH, novel non-invasive point-of-care technologies can identify patients correctly, thereby further improving the safety and accuracy of a multifaceted approach to the initial diagnosis of PH. With the growing risk of PH in our population, a tool for providing noninvasive assessment of pulmonary hemodynamics is essential today.

There is a need for noninvasive cardiopulmonary health assessment and monitoring systems and methods that can provide timely and enhanced patient experience by enabling telehealth remote patient monitoring (RPM) or at-home self-monitoring of heart and lung conditions. There is also a need for an accurate and affordable screening tool that enables early identification and treatments of certain serious conditions and increase survival rate. Therefore, there is a need for systems and methods that can effectively measure and assess one or more different cardiopulmonary conditions. There is also a need to integrate such cardiopulmonary health assessment systems and methods with computing devices that continue to grow in computing capability and power.

The exemplary embodiments herein provide methods and systems based on a technique of separating, identifying, extracting, and marking cardiac events from noninvasively captured physiological signals related to hemodynamics and assessing the cardiopulmonary health in a subject, specifically left ventricular end-diastolic pressure (LVEDP) and pulmonary capillary wedge pressure (PCWP).

The features or parameters can be ultimately used in a trained model or classifier (e.g., a trained machine-learned classifier) to estimate metrics associated with the physiological state of a subject, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

The cardiohemic system models the kinematics of the motion of the lumped system which consists of the ventricle, its contents, and surrounding structures such as walls, valves, and blood, displaced by inertial forces, creating fluid deceleration in the ventricle and its causal relation to the oscillations (vibrations and rotational inertia) due to abrupt mechanical opening and closing of the valves during each cardiac cycle.

Examples of vibration objects are mitral valve opening (MVO) and closing (MVC), aortic valve opening (AVO) and closing (AVC), pulmonary valve opening (PVO) and closing (PVC), tricuspid valve opening (TVO) and closing (TVC), and heart wall motions. A portion of the energy produced by these vibrations lies in the infra-sound range (generally below 20 Hz), which falls in the inaudible and low-sensitivity human hearing range. Another portion of the energy produced by these vibrations falls in the audible hearing range. Vibration transducers or accelerometers placed on the thoracic region or chest capture these vibrations from both of these ranges. Data can be obtained using a tri-axial accelerometer or multiple tri-axial accelerometers placed on different points of torso.

Similarly, other cardiac waveform sensor modalities capture signals correlated with the cardiohemic events, including but not limited to electrocardiogram (ECG), impedance cardiography (ICG), phonocardiography (PCG), photoplethysmography (PPG), seismocardiography (SCG), ballistocardiography (BCG), gyrocardiography (GCG), along with echocardiography (echo).

The opening and closing of the atrioventricular (AV) valves are dependent on pressure differences between the atria and ventricles. When the ventricles relax, atrial pressure exceeds ventricular pressure, the AV valves are pushed open and blood flows into the ventricles. Conversely, when the ventricles contract, ventricular pressure exceeds atrial pressure causing the AV valves to snap shut.

The semilunar valves (pulmonary valve and aortic valve) are one-way valves that separate the ventricles from major arteries. The aortic valve separates the left ventricle from the aorta, while the pulmonary valve separates the right ventricle from the pulmonary artery. As the ventricles contract, ventricular pressure exceeds arterial pressure, the semilunar valves open and blood is pumped into the major arteries. Conversely, when the ventricles relax, arterial pressure exceeds ventricular pressure and the semilunar valves snap shut. This is due to the elevated pressures in the aorta and the pulmonary artery pushing the blood back toward the ventricles to close the semilunar valves.

By analyzing one or a plurality of the physiological signals capture noninvasively, including, but are not limited to ECG, ICG, PCG, SCG, BCG, GCG, PPG, and echo, the precise valve timings and durations in each cardiac cycle are identified, which exhibit causal relationship to the pressure-volume (PV) loop, and extracted to assess the filling times and determine presence of elevated LVEDP or PCWP.

The present disclosure is best understood with reference to the detailed figures and description set forth herein. In the following description, various embodiments will be described. For purpose of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

The described embodiments may be implemented manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by practitioners of the art to which the embodiments pertain. Persons skilled in the art will envision many other variations that are within the scope of the claimed subject matter.

The exemplary embodiments of the invention are directed to a system and method for non-invasively determining at least one of left ventricular end diastolic pressure (LVEDP) or pulmonary capillary wedge pressure (PCWP) in a subject's heart.

The present subject matter is based on the fact that a healthy cardiopulmonary system in the human body represents a delicate coupling between heart pumping characteristics, timings, fluid volume status, and filling pressures. The optimum coupling is impaired by aging, arterial and cardiovascular diseases such as hypertension and heart failure.

Hemodynamic evaluation is an essential component in diagnosing cardiovascular disorders and managing patient care.

The left ventricular pressure-volume loop provides a simple yet extremely useful framework for understanding cardiac mechanics, shown in the chart 1400 of FIG. 14.

The cardiac time intervals (CTIs) are shown the chart 1500 of in FIG. 15.

The slope of the end-systolic pressure-volume relationship (ESPVR) indicates end-systolic elastance, an index of contractility; and the slope of the end-diastolic pressure-volume relationship (EDPVR) indicates ventricular elastance or stiffness (the reciprocal of ventricular compliance). Several physiological relevant hemodynamic parameters can be determined from this, specifically the cardiac events corresponding to the opening and closing of the heart valves: Mitral Valve Closing (MVC), Mitral Valve Opening (MVO), Aortic Valve Opening (AVO) and Aortic Valve Closing (AVC). Ventricular filling occurs along the EDPVR, a monotonically increasing curve: pressure is directly proportional to the volume of blood in the left ventricle, and volume is proportional to filling time.

Under normal conditions (shown by loop 1404 in FIG. 14), a longer diastolic filling time (DFT), computed as the cardiac time interval (CTI) from MVO to the MVC or

DFT≙MVO to MVC

correlates with a higher LVEDP. In patients with HFpEF (shown by dashed loop 1402 in FIG. 14), stiffness is increased compared to age-matched control subjects (the heart muscle also stiffens with age), and the EDPVR is effectively shifted up (from the curve 1408 to the curve 1410) with a steeper slope. Consequently, the same incremental change in volume yields a higher filling pressure, and a smaller increase in volume or DFT results in a higher LVEDP than normal. Thus, the DFT cardiac time interval provides an alternative method, for accurate and absolute estimation of LVEDP, to that of using left heart catheterization.

In another embodiment, the other common cardiac time intervals (CTIs) associated with the four valve events are (shown in FIG. 15):

Left Ventricular Ejection Time: (LVET) [symbol] AVO to AVC

Isovolumic Contraction Time: (IVCT) [symbol] MVC to AVO

Isovolumic Relaxation Time: (IVRT) [symbol] AVC to MVO

Diastolic time: DT [symbol] (AVC to MVC)

Systolic time: ST [symbol] (MVC to AVC)

and the parameters can be combined to form derivative CTIs or ratios directly or normalized by the heat rate (HR) in beats per minute.

In another embodiment, the diastolic to systolic duration ratio (D/S ratio or DSR) is another key index for identifying diastolic heart failure and defined as:

DSR [symbol] (AVC to MVC)/(MVC to AVC).

For HFrEF patients (shown by dashed loop 1406 in FIG. 14), the ventricle dilates with reduced contractility, resulting in a higher left ventricular end-diastolic volume (LVEDV) and pressure (LVEDP). Assuming the same stiffness or EDPVR curve, the pressure-volume loop is effectively shifted to the right (from the loop 1404 to the dashed loop 1406). For HFrEF patients, a reduction in diastolic duration is compensated by a reduced systolic duration because the ejection time is reduced, resulting in a maintained DSR.

Additionally or alternatively, to distinguish preserved vs. impaired LV systolic function, the systolic time ratio (STR) can be used, computed using the following CTIs:

STR [symbol] AVO/(AVO to AVC)=PEP/LVET

where the pre-ejection period (PEP) is equivalent to the AVO with its starting point referenced at the onset of the QRS complex. To account for changes in heart rate (HR), a heart rate normalized, systolic time ratio index (STRi) can be used.

In an embodiment, from the cardiac mechanics, a key hemodynamic feature for accurate measurement of elevated LVEDP levels is DSR.

In an embodiment, to furthermore distinguish between diastolic and systolic dysfunction, both DSR and STR can be used.

In an embodiment, the CTI based features such as DSR and STR can be accurately obtained inexpensively and noninvasive using a single or a plurality of sensor modalities (which include but not limited to one or more sound transducers, accelerometers, gyroscopes, bioreactance sensors, and ECG electrodes) in any combination of physiological signals such as impedance cardiography (ICG), phonocardiography (PCG), seismocardiography (SCG), gyrocardiography (GCG), electrocardiogram (ECG), and as well as noninvasive measurements of the opening and closure of the aortic and mitral valves using different ultrasound modalities, i.e., echocardiogram (echo), such as M-mode, Doppler flow imaging, Tissue Doppler Imaging (TDI) or speckle tracking strains.

In an embodiment, AVC can be determined noninvasively using heart sounds either obtained from an acoustic sensor such as a digital stethoscope, i.e., PCG, additionally or alternatively from an accelerometer which measures the cardiac mechanical processes including cardiac muscle contraction, cardiac valve movement, blood flow turbulence, and momentum changes, i.e. SCG, or from angular displacement using gyroscope, i.e., GCG.

In an embodiment, the second heart sound (S2) consists of two major components: AVC or A2 and pulmonic valve closure (P2). The peak of the S2 envelope correlates to the onset of the aortic valve closure in a stable and consistent mechanical fashion, i.e., the S2 is caused by the AVC. This relationship can be established from training data consisting of paired S2 peak timing and AVC timing obtained from the gold standard echocardiography and is generalizable across patients for the same sensor modality. Manufacturing differences in sensors result in different filtering delays that require recalibration, i.e., the specific relationship is dependent of the sensor modality and settings. In one specific embodiment, a simple relationship is established using linear regression.

In an alternative embodiment, applying machine learning techniques, such as a neural network of a single or multiple layers may be used to output the AVC from either S2 peak timing or directly from the raw recording. Note that the embodiments herein are not limited to the sensor modalities or methods used to obtain the CTIs and all CTI derived features such as LVET, STR, DSR, IVRT, LVET, IVCT, from noninvasive physiological signals.

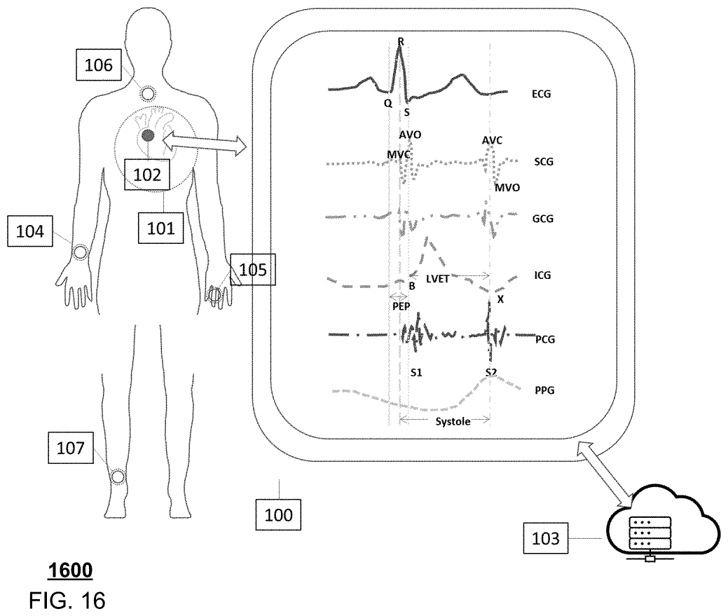

In an embodiment, to find the S2 envelope peak, we first segment cardiac signal into individual beats using synchronized electrocardiogram (ECG) recording and its fiducial points (FIG. 16). For each beat, the ECG and PCG (or SCG) are aligned using the onset of the QRS complex or u-point. The vibrational waveform (PCG or SCG) is bandpass filtered to emphasize the high frequency components, e.g., 50-240 Hz, then its energy envelope is used to find the peak amplitude and its corresponding time stamp. To further narrow the search window for potential S2 sound, we can leverage physiological knowledge, i.e., S2 occurs past the S-point of the QRS complex, around the T-wave, and marks the end of systole or the beginning of diastole. The diastole is the longer of the two phases, so that the heart can rest between contractions. We can use the beat duration (computed from R-R intervals) to mark the potential range of the two phases in each beat.

The exemplary embodiments of the system and method proposed here are shown in FIG. 16. 100 is an embedded platform which can be any smart processing platform with digital signal processing capabilities, application processor, data storage, display, input modality like touch-screen or keypad, microphones, speaker, Bluetooth, and connection to the internet via WAN, Wi-Fi, Ethernet or USB. This embodies custom embedded hardware, smartphone, iPad-like and iPod-like devices. 101 in FIG. 16 is the auditory scene at the chest locations. 102 in FIG. 16 are the transducer or transducer array used to captures the heart signal and the wearable microprocessor hardware with digital signal processing capabilities, application processor, Analog to digital frontend, data storage, input modality like buttons, and wireless connection via Bluetooth, Bluetooth low energy, near field communication transceiver, Wi-Fi, Ethernet or USB. 102 in FIG. 16 comprises of the signal processing module on the wearable that captures synchronized sensor data from the transducer array. Saves it to memory and communicates with 100 for data transfer. 100 in FIG. 16 is the module that allows a user to visually see the individual streams and information from the heart valves, in an embodiment this could be a connected display or any other modality of display. 102 are multiple sensor transducers that capture the composite signal; comprising of the electrocardiogram signals, heart sounds, lung sounds and snoring sounds. 102 is the wearable hardware that synchronously collects the signals across the transducers and is responsible for the analog to digital conversation, storage and transmission to the portable unit. 103 in FIG. 16 is the module that calculates vital sign from the input sensor stream coming from 102 for the Heart rate, breathing rate, EKG signal, skin temperature, and associated vitals. It encrypts the raw sensor data for transmission to the cloud computing module 103. Its also communicates with a dashboard on 103 for data exchange, login, alerts, notifications, display of processed data. 103 in FIG. 16 is the cloud module that processes the individual streams for eventual source separation, identification and marking of the heart valve signals.

The exemplary embodiments of the system and method proposed here include a portable electronic device or an embedded hardware system and a plurality of electronic components, the main elements required to capture body cardiohemic signals are the sensor units that capture, digitize, and process signals for noise reduction, filtering, and amplification.

The exemplary embodiments of the system and method proposed here provide a microcontroller that transmits the electrophysiological data received from the plurality of ECG electrodes, the sound transducer, and the vibrational and rotational sensors to at least one of the portable electronic device (PED) and a computing device. The PED and computing device are configured to: receive, in one or more temporal windows, a representation of data from one or more of the following when positioned against the thoracic cavity of the user: one or a plurality of ECG, ICG, PCG, SCG, BCG, GCG, PPG, and echo; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the cardiac or pulmonary health of the user and/or estimate a progression of a cardiac or pulmonary health condition.

For the exemplary embodiments of the system and method proposed here, the PED may be held against the thoracic cage of the user, the back of the user, and/or the sternum of the user to emit sound into the body of the user.

For the exemplary embodiments of the system and method proposed here may be a wearable, include, but not limited to a patch, ring, belt, band, bracelet, necklace, or clothing for prolonged capturing, monitoring, and transmission of electrophysiological data. 104-107 in FIG. 16 illustrates possible locations of device placement.

For the exemplary embodiments of the system and method proposed here, other noninvasively obtained biological information are used to supplement the captured signal such as age, height, weight, and sex. These are just examples of biological information and any other types of biological information can be used within contemplation of the embodiments and are not limited to the examples disclosed.

Biological sex plays an important role in cardiac physiology and cardiovascular function, in particular, women significantly outnumber men in HFpEF by a ratio of 2:1. Left ventricular dimensions and function are distinctly different in healthy women and men, even after taking body size into consideration: women have smaller LV chambers, thus lower stroke volumes, but maintains a similar cardiac output with a higher resting heart rate, higher systolic and diastolic LV elastance (stiffness) at a given age, and aging accentuates these differences, with a steeper increase in LV elastance. Women are also significantly more susceptible (4 times more likely) to idiopathic pulmonary arterial hypertension (PAH). This strongly suggests underlying sex differences in pulmonary vascular function, remodeling, and reactivity. In one study, 82% of patients with HFpEF and PH are female, compared with 58% of those without PH. Furthermore, the sex differences in LV development diverges with age. Only a small sex difference (mean=6%) in LV mass existed before age 12, compared to 25% to 38% greater mass in men (p-value<0.02 to p-value<0.0001) in all older strata, paralleling the sex differences in height and weight. As patients age (free of clinical cardiovascular disease at baseline), the LV responds differently in its mass and volume between men and women (8.0 and −1.6 g per decade, respectively), although both experiences increased concentric LV remodeling: LV end-diastolic volume decreases −9.8 and −13.3 mL per decade, respectively, stroke volume decreased −8.8 and −8.6 mL per decade, respectively, and mass-to-volume ratio increased 0.14 and 0.11 g/mL per decade, respectively. Change in LV mass correlates positively with systolic blood pressure (sBP) and body mass index (BMI), and negatively with treated hypertension and high-density lipoprotein cholesterol level. These age and sex-related differences play an important role in our PH detection algorithm.

In an embodiment, due to the significant sex differences for LVEDP assessment, a different model is trained for male and female patients to optimize the performance. Furthermore, to standardize and normalize the LVEDP performance across different patients and to derive a generalizable measure, we incorporate the following, easily obtainable, noninvasive, clinical and demographic characteristics of the patients: age, height, and weight.

For the exemplary embodiments of the system and method proposed here may be powered by a battery or through energy harvesting.

Some embodiments of the instant invention are directed to a system for non-invasively determining at least one of left ventricular end diastolic pressure (LVEDP) or pulmonary capillary wedge pressure (PCWP) in a subject's heart, using physiological knowledge of the cardiac mechanics captured by the sensors.

In an alternative embodiment, instead of calculating the CTIs, relevant features are automatically learned from the raw signals without manual feature engineering using machine learning techniques, including deep multimodal representational learning.

The exemplary embodiments of the system and method proposed here provide an analysis algorithm to non-invasively measure a physical property of said subject's heart that is correlated with said subject's heart beat so as to provide timing signals comprising timing information with respect to heartbeat cycles, and wherein said signal processor is configured to receive said peripheral pressure signals and said timing signals and to non-invasively determine an estimated value of said subject's LVEDP or PCWP based at least partially thereon.

The exemplary embodiments of the system and method proposed here provide an analytics engine that may use one or more artificial intelligence based algorithms, including trained algorithms such as machine learning algorithms based on neural networks or other similar technologies, to identify new patterns in time series data. For example, a trained artificial intelligence (AI) algorithm may be a trained machine learning algorithm that may be implemented by a deep learning approach.

For the exemplary embodiments of the system and method proposed here, the trained artificial intelligence algorithm may take as input recorded biological sensor data in order to determine a health condition of a patient, and in some examples, determine a specific treatment. For example, the biological sensor data may be input into the trained AI algorithm. Additional information such as age, gender, recording position, weight, or organ type may be inputted into the trained AI algorithm. The trained AI algorithm may output a likelihood of a pathology or disease, a disease severity score, or a healthy state, for example. In some cases, the trained AI algorithm may be used to analyze a subset of biological sensor data, such as audio data, ECG data, or both the audio and the ECG data.

For the exemplary embodiments of the system and method proposed here, as a non-limiting example, a trained AI algorithm may take as input time-synchronized time series data that includes ECG data and heart sound data, obtained from an ECG transducer and audio transducer of the monitoring device, process the input ECG and heart sound data, and output a heart condition of the patient. During the processing, the AI algorithm may extract features of the input ECG and heart sound data and evaluate the extracted features with respect to plurality of annotated data sets comprising time synchronized ECG data and heart sound data, wherein the plurality of annotated data sets (labelled by domain experts) are stored in a database of the analytics engine, the plurality of annotated data sets obtained from a plurality of subjects. For example, the features of the audio data, the ECG data, or a combination of the audio and ECG data can be used to determine a heart condition of a subject. In some examples, the trained AI algorithm may compare and identify a number of examples from the annotated data sets that are closest in terms of a plurality of features of ECG data and/or audio data to a recorded ECG or audio data. The identified datasets that include features highly similar to the recorded data that is currently analyzed may be provided as output to assist the clinician in diagnosis.

For the exemplary embodiments of the system and method proposed here, additionally, the trained artificial intelligence algorithm may analyze the input ECG and heart sound data with a plurality of ECG and hear sound data of the same patient obtained at various times prior to the input data to determine progression of the heart condition. The role of the server 180 in handling recording and AI requests is discussed in further detail below.

For the exemplary embodiments of the system and method proposed here, the trained AI algorithm may be implemented by a deep learning model. The deep learning model may be trained by a supervised method, on deep convolutional neural networks (CNNs). A training data set for supervised learning problem may biological sensor data reviewed and labeled by domain experts (e.g., physicians, clinicians, etc.). In some examples, the training may be implemented by an unsupervised method or a semi-supervised method based on deep CNNs.

For the exemplary embodiments of the system and method proposed here, the algorithm may be trained by a training set that is specific to a given application, such as, for example classifying a state or condition (e.g., a disease). The training data set for a heart condition may be different from a training data set for a lung condition, for example. In some examples, the training set (e.g., type of data and size of the training set) may be selected such that, in validation, the algorithm yields an output having a predetermined accuracy, sensitivity and/or specificity (e.g., an accuracy of at least 90% when tested on a validation or test sample independent of the training set).

For the exemplary embodiments of the system and method proposed here, further, during the processing by the trained algorithm, data from different time periods may be compared, whereby a first set of biological data of a patient over a first time period may be processed and/or compared with and a second set of biological data of the patient over a second time period. As a non-limiting example, a patient with an arrhythmia may transmit recorded ECG data to a server over a period of two years, and for an annual remote consultation, a specialist may process the ECG data recorded over the first year with the ECG data recorded over the second year via a program that uses an expert system or trained algorithm to identify trends, which may determine that the patient's arrhythmia is gradually changing.

For the exemplary embodiments of the system and method proposed here, patient biological data that is recorded and transmitted to the computing system may be processed on its own, and/or along with patient data previously transmitted by the patient, or it may be processed (e.g., compared) with control samples and/or patient data transmitted by other patients. For example, during a single remote consultation with a patient, a remote clinician may run an analytics program on ongoing live stream data, to determine a health condition of the patient. Additionally or alternatively, the patient data is compared with historical data from a variety of anonymized patients, thereby determining that the patient is suffering from a particular kind of arrhythmia, and/or the patient's data may be compared with historical data for the patient over the duration of the patient's relationship with the care facility to evaluate a patient's disease progression.

The exemplary embodiment of system and method described is the development on an embedded hardware system, the main elements required to capture body sounds are the sensor unit that captures the body sounds, digitization, and digital processing of the body sounds for noise reduction, filtering and amplification.

It will be apparent to those skilled in the art that various modifications may be made in the present embodiments without departing from the spirit or scope of the embodiments. Thus, it is intended that the present embodiments cover the modifications and variations provided they come within the scope of the method and system described and their equivalents.

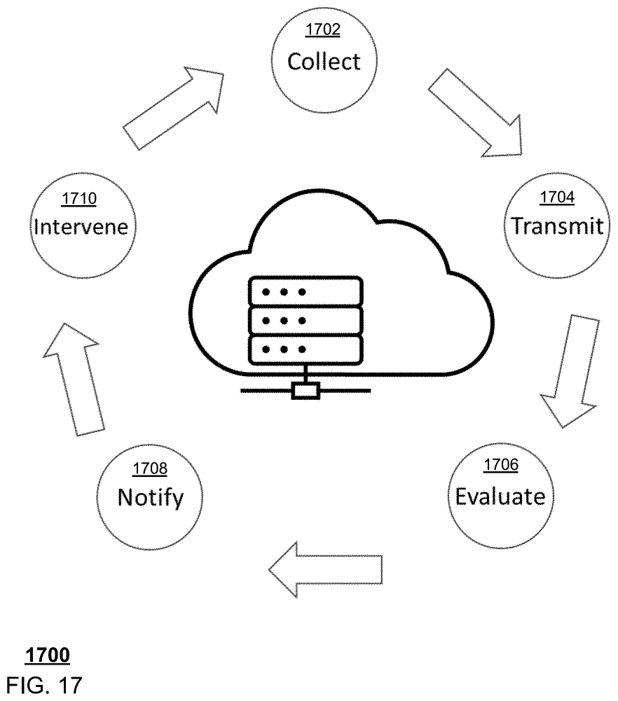

FIG. 17 illustrates a complete Remote Patient Monitoring (RPM) system 1700 for telehealth using the exemplary embodiment of system and method described. In such a system, a patient can wear a device such as described with respect to FIG. 1A, 1B, 1C, or 16 and collect data at 1702, transmit the data at 1704 for evaluation at 1706 and appropriately notify the patient at 1708 or even intervene at 1710 when necessary as the system can continue to cycle after notification or intervening to periodically, randomly, or continuously collect data at 1702. The described noninvasive cardiopulmonary health assessment and monitoring systems and methods can provide effective at-home self-assessment or used in an integrated telehealth RPM system, allowing health professionals to provide timely interventions and improve the delivery of care.

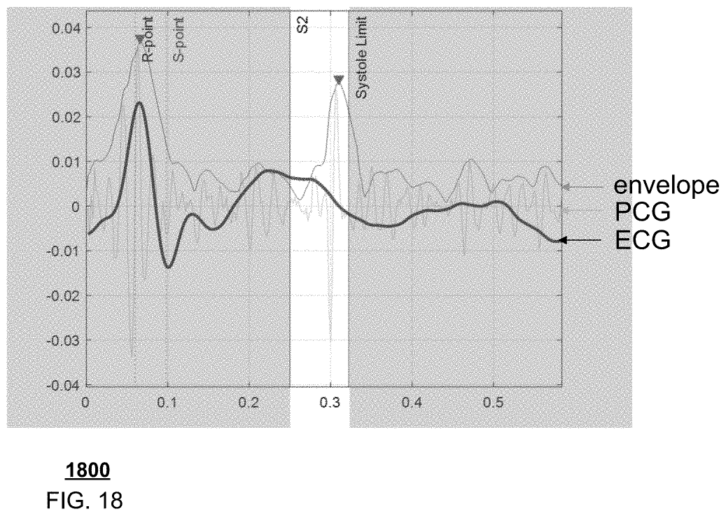

The chart 1800 of FIG. 18 illustrates the marking of vibration objects in accordance with an embodiment. Such vibration objects can be used as reference points relative to other data collected in accordance with the embodiments. In one embodiment, the cardiac time interval for Aortic Valve Closing (AVC) can be detected noninvasively using heart sounds either obtained from an acoustic sensor such as a digital stethoscope, i.e., phonocardiography (PCG), additionally or alternatively from an accelerometer which measures the cardiac mechanical processes including cardiac muscle contraction, cardiac valve movement, blood flow turbulence, and momentum changes, i.e. seismocardiography (SCG). The second heart sound (S2) consists of two major components: AVC or A2 and pulmonic valve closure (P2). The peak of the S2 envelope correlates to the onset of the aortic valve closure in a stable and consistent mechanical fashion, i.e., the S2 is caused by the AVC. This relationship can be established from training data consisting of paired S2 peak timing and AVC timing obtained from the gold standard echocardiography and is generalizable across patients for the same sensor modality. Manufacturing differences in sensors result in different filtering delays that require recalibration, i.e., the specific relationship is dependent of the sensor modality and settings. In one specific embodiment, a simple relationship is established using linear regression. In an alternative embodiment, a neural network of a single or multiple layers may be used to output the AVC from S2 peak timing. Note that the embodiments herein are not limited to the sensor modality or the method used to obtain AVC from S2 peak timing. In one embodiment, to find the S2 envelope peak, we first segment cardiac signal into individual beats using synchronized electrocardiogram (ECG) recording and its fiducial points (FIG. 18). For each beat, the ECG and PCG (or SCG) are aligned using the onset of the QRS complex or u-point. The vibrational waveform (PCG or SCG) is bandpass filtered to emphasize the high frequency components, e.g., 50-240 Hz, then its energy envelope is used to find the peak amplitude and its corresponding time stamp. To further narrow the search window for potential S2 sound, we can leverage physiological knowledge, i.e., S2 occurs past the S-point of the QRS complex, around the T-wave, and marks the end of systole or the beginning of diastole. The diastole is the longer of the two phases, so that the heart can rest between contractions. We can use the beat duration (computed from R-R intervals) to mark the potential range of the two phases in each beat.

It will be apparent to those skilled in the art that various modifications may be made in the present invention without departing from the spirit or scope of the invention. Thus, it is intended that the present invention cover the modifications and variations of this invention provided they come within the scope of the method and system described and their equivalents.

What is claimed is:

1. A method for noninvasive evaluating and monitoring of a contractility feature, comprising:
obtaining one or more signals using one or more noninvasive sensors or transducers that provide a measure of one or more physiological effects that are correlated with cardiopulmonary functions, the measure based on quantitative measurements of heart and lung related parameters and cardiac events for diagnostic and therapeutic purposes;
computing, by at least one processor, an elevated left ventricular end-diastolic pressure (LVEDP) or an pulmonary capillary wedge pressure (PCWP) based on explicit or implicit time features and waveform features of the one or more signals, wherein the LVEDP or the PCWP are computed at a rest assessment of hemodynamic performance.

2. The method of claim 1, wherein the contractility feature comprises cardiac time intervals, and the method further comprising calculating, by the at least one processor, cardiac time intervals based on cardiac waveform data.

3. The method of claim 2, wherein the at least one processor calculates the LVEDP or the PCWP as a function of the cardiac time intervals, and an electrocardiogram (ECG).

4. The method of claim 1, further comprising calculating the contractility feature based on a cardiac waveform and an electrocardiogram (ECG), wherein the contractility feature comprises a derivative of the cardiac waveform data.

5. The method of claim 1, wherein the explicit or implicit time features and waveform features of the one or more signals comprises cardiac vibrations, and the method further comprising collecting a cardiac vibration waveform.

6. The method of claim 1, further comprising correcting the calculation of the LVEDP or the PCWP for valvular diseases based on the cardiac vibration waveform.

7. The method of claim 1, further comprising monitoring, by the at least one processor, the contractility feature by an imaging modality including at least one cardiac magnetic resonance imaging or an echocardiogram.

8. The method of claim 1, wherein the monitoring of the contractility Feature is based on an electrocardiogram (ECG) and on at least one of: impedance cardiography (ICG), phonocardiography (PCG), photoplethysmography (PPG), seismocardiography (SCG), ballistocardiography (BCG), gyrocardiography (GCG), or echocardiography (echo).

9. The method of claim 1, wherein monitoring the contractility feature is based on at least one of: calculating a surrogate ejection fraction (EF) from non-invasively measured cardiac time intervals or a cardiac waveform.

10. An apparatus for approximation of left ventricular end diastolic pressure (LVEDP) or an pulmonary capillary wedge pressure (PCWP), comprising:
a non-invasive cardiac waveform sensor;
at least one of a non-invasive electrocardiogram (ECG) sensor
one or more processors coupled to the non-invasive cardiac waveform sensor, the at least one non-invasive ECG sensor, and a memory, wherein the memory holds computer instructions that when executed by the one or more processors cause the apparatus to perform:
receiving cardiac waveform data from the non-invasive cardiac waveform sensor configured for coupling to a patient;
receiving electrocardiogram (ECG) data via the non-invasive ECG sensor;
determining at least one of a pre-ejection period (PEP) or an isovolurnic contraction time (IVCT), based on simultaneous received portions of the cardiac waveform data and the ECG data from the non-invasive cardiac waveform sensor;
calculating an LVEDP or a PCWP based on a contractility feature and at least one of the cardiac time intervals; and
encoding the LVEDP or the PCWP as digital data for at least one of storage, transmission, or human-comprehensible output.

11. The apparatus of claim 10, wherein the memory holds instructions for calculating the contractility feature comprising cardiac time intervals based on the cardiac waveform data.

12. The apparatus of claim 10, wherein the memory holds instructions for calculating the contractility feature based on at least one of: electrocardiogram (ECG), impedance cardiography (ICG), phonocardiography (PCG), photoplethysmography (PPG), seismocardiography (SCG), ballistocardiography (BCG), gyrocardiography (GCG), or echocardiography (echo).

13. The apparatus of claim 10, wherein the memory holds instructions for calculating the contractility feature based on at least one of: calculating a surrogate ejection fraction (EF) from non-invasively measured cardiac waveform.

14. An apparatus for approximation of left ventricular end diastolic pressure (LVEDP), comprising:
a non-invasive cardiac waveform sensor;
at least one of a non-invasive electrocardiogram (ECG) sensor or heart vibration waveform sensor;
at least one processor coupled to the non-invasive cardiac waveform sensor, the at least one of the non-invasive ECG sensor or heart vibration waveform sensor, and a memory, wherein the memory holds computer instructions that when executed by the at least one processor cause the apparatus to perform:
receiving cardiac waveform data from a non-invasive sensor coupled to a patient and at least one of electrocardiogram (ECG) data or heart vibration waveform data;
synchronizing, the cardiac waveform data and the at least one of electrocardiogram (ECG) data or heart vibration waveform data; and
calculating an LVEDP based on time features and waveform features of the cardiac waveform data and the at least one of electrocardiogram (ECG) data or heart vibration waveform data.

15. The apparatus of claim 14, wherein the memory holds instructions for encoding the LVEDP as digital data for at least one of storage, transmission, or human-comprehensible output.

16. The apparatus of claim 14, wherein the memory holds instructions for determining, at least one of a pre-ejection period (PEP) or an isovolumic contraction time (IVCT), based on simultaneous portions of the cardiac waveform data and at least one of the ECG data or the heart vibration waveform data.

17. The apparatus of claim 14, wherein calculating the LVEDP or a PCWP based on an implicit extraction of cardiac time intervals using machine learning or deep learning to automatically map a input signal into a desired output for LVEDP or PCWP.

18. The apparatus of claim 17, wherein the memory holds instructions for correcting the calculating of the LVEDP for valvular diseases based on the cardiac waveform

19. The apparatus of claim 14, wherein calculating the LVEDP based on the time features and waveform features of the cardiac waveform data and the at least one of electrocardiogram (ECG) data or heart vibration waveform data is based on or supplemented with at least one of: calculating the diastolic filling time (DFT), calculating the diastolic time (DT), calculating the systolic time (ST), a ratio or combination of cardiac time intervals, or calculating a surrogate ejection fraction (EF) from non-invasively measured cardiac waveform.

20. The apparatus of claim 14, wherein calculating the LVEDP based on the time features and waveform features of the cardiac waveform data and the at least one of electrocardiogram (ECG) data or heart vibration waveform data is based on or supplemented with at least one of a noninvasively obtained cardiac waveform or biological information.
